(19) United States
(12) Patent Application Publication
(10) Pub. No.: US 2018/0185448 A1
(43) Pub. Date: Jul. 5, 2018

(54) COMPOSITION AND METHOD FOR INDUCING ANTI-INFLAMMATORY RESPONSE

(71) Applicant: Vital Therapies, Inc., San Diego, CA (US)

(72) Inventor: Robert A. Ashley, Encinitas, CA (US)

(21) Appl. No.: 15/580,670

(22) PCT Filed: Jun. 14, 2016
(86) PCT No.: PCT/US16/37402

§ 371 Date: Dec. 7, 2017

Related U.S. Application Data
(60) Provisional application No. 62/175,827, filed on Jun. 15, 2015.
(60) Provisional application No. 62/190,067, filed on Jul. 8, 2015.

(51) Int. Cl. A61K 38/20; A61K 38/19; A61K 31/739; A61P 1/16; A61P 29/00; C12N 5/071; C12N 5/09

(52) U.S. Cl. CPC A61K 38/204; A61K 38/2006; A61K 38/191; C12N 5/0693; A61P 1/16; A61P 29/00; C12N 5/067; A61K 31/739

(57) ABSTRACT

The present invention relates generally to metabolic detoxification, and more particularly to a composition and method for inducing an anti-inflammatory response in a cell, as well as treating disease in a subject.

### CROSS REFERENCE TO RELATED APPLICATIONS

This application claims the benefit of priority under 35 U.S.C. § 119(e) of U.S. Provisional Patent Application Ser. No. 62/175,827, filed Jun. 15, 2015, and U.S. Provisional Patent Application Ser. No. 62/190,067, filed Jul. 8, 2015, the entire contents of which are incorporated herein by reference in their entireties.

### BACKGROUND

### Field of Invention

The present invention relates generally to cell biology, and more particularly to a composition and method for inducing an anti-inflammatory response in a cell, as well as treating disease in a subject.

### Background Information

Various agents, including but not limited to bacteria, viruses, physical injury, chemical injury (for example, alcohol, drugs and the like), cancer, chemotherapy, and radiation therapy, can, depending on the specific agent and the genetic makeup of the animal exposed to it, cause direct damage to cells and tissue or create an environment of prolonged and excessive inflammation. Under normal conditions, inflammation is a process that helps an animal recover from injury. Acute inflammation is the initial response of a tissue to harmful stimuli. It involves a complex, highly regulated process that begins when cells present in the injured tissue, including macrophages, dendritic cells, histiocytes, Kupffer cells, and mastocytes, sense molecules associated with the injury and become activated. Upon activation, these cells release inflammatory mediators, such as vasodilators. The vasodilators induce increased blood flow and permeability of the blood vessels in the vicinity of the injury. This, in turn, results in the increased movement of plasma and leukocytes (including neutrophils and macrophages) from the blood into the injured tissue. Because inflammatory mediators are, in general, rapidly degraded, acute inflammation requires constant stimulation in order to be sustained. As a result, acute inflammation ends once the harmful stimulus is removed.

Chronic inflammation is believed to be a contributing factor to many widespread and debilitating diseases, including liver diseases, such as hepatitis, cirrhosis and fatty liver disease, heart disease, cancer, respiratory disease, stroke, neurological diseases such as Alzheimer's disease, diabetes, and kidney disease. The result of chronic inflammation is the destruction of normal tissue and its replacement with collagen-rich connective tissue. Collagen-rich connective tissue, also known as scar tissue, exhibits diminished tissue function as compared to normal tissue. Persistent and prolonged formation of scar tissue, in turn, leads to fibrosis. Fibrosis is among the common symptoms of diseases affecting the lungs, skin, liver, heart, and bone marrow, and is a critical factor in diseases such as idiopathic pulmonary fibrosis, scleroderma, keloids, liver cirrhosis, myocardial fibrosis, diabetic kidney disease, myelodysplastic syndrome, and other disorders.

Studies of chronic inflammation and fibrosis have indicated that, regardless of the activating agent and the tissue affected, a common network of signaling proteins tend to function together to establish the pro-inflammatory state. This network of signaling proteins includes a number of different cytokines, cytokine receptors, transcription factors, and the like, including IL-1β and IL-6.

The processing of blood has been performed to remove a variety of blood constituents for therapeutic purposes including inflammatory liver diseases, such as hepatitis. Examples of blood processing methods include hemodialysis that allows to remove metabolic waste products from the blood of patients suffering from inadequate kidney function. Blood flowing from the patient is filtrated to remove these waste products, and then returned to the patient. The method of plasmapheresis also processes blood using tangential flow membrane separation, to treat a wide variety of disease states. Membrane pore sizes can be selected to remove the unwanted plasma constituents. Blood can be also processed using various devices utilizing biochemical reactions to modify biological constituents that are present in blood. For instance, blood components such as bilirubin or phenols can be gluconized or sulfated by the in vitro circulation of blood plasma across enzymes that are bonded to membrane surfaces.

Presently used technologies are generally deficient with respect to supporting patients with compromised liver function, for example. Conventional systems and methods suffer from various problems associated with sustaining such patients until a suitable donor organ can be found for transplantation or until the patient's native liver can regenerate to a healthy state.

Inflammatory diseases such as hepatitis caused by alcohol, drugs or toxins are characterized by elevated levels of IL-1 β, IL-6, and TNFα and an inflammatory cascade in which hepatocytes up-regulate acute-phase proteins (APP). Thus patients in need of blood detoxification treatment typically exhibit elevated levels of IL-1 β, IL-6, and TNFα. Anti-TNFα or steroidal therapies have not demonstrated clinical benefit.

Despite growing knowledge about conditions that involve excessive inflammation, treatments for such conditions remain elusive. Many drugs and other substances have been shown to have anti-inflammatory activity, either in vitro or in vivo, but for many indications caused or potentiated by inflammation, there are still no therapies. In addition, many anti-inflammatory therapies are associated with harmful side effects. Thus a need exists for more advanced compositions and methods to treat inflammatory diseases.

### SUMMARY OF THE INVENTION

In one aspect, the present disclosure provides a composition for inducing an anti-inflammatory response in a cell. The composition includes one or more pro-inflammatory molecules, such as lipopolysaccharide (LPS) or pro-inflammatory cytokines Interleukin-6 (IL-6) and Interleukin-1 beta (IL-1β), wherein the anti-inflammatory response comprises increased expression of anti-inflammatory factors, such as anti-inflammatory mediator proteins α-1-antitrypsin (AAT) and Interleukin-1 receptor antagonist (IL-1Ra).

In another aspect, the present disclosure provides a method of inducing an anti-inflammatory response, or inhibiting an inflammatory response, in a cell. The method includes contacting the cell with a composition of the disclosure, thereby inducing an anti-inflammatory response, or inhibiting an inflammatory response, in the cell.

In still another aspect, the present disclosure provides a method of treating a disease or disorder in a subject. The method includes administering a composition of the disclosure to the subject, thereby treating the disease or disorder.

In yet another aspect, the present disclosure provides a qualified C3A cell line derived from a parental C3A cell line, wherein cells of the cell line exhibit increased expression of anti-inflammatory factors, such as anti-inflammatory mediator proteins AAT and IL-1Ra, in response to one or more pro-inflammatory molecules, such as LPS or pro-inflammatory cytokines IL-6 and IL-1β.

### BRIEF DESCRIPTION OF THE DRAWINGS

FIG. 1 is a graphical plot depicting data relating to an embodiment of the invention.

FIG. 2 is a graphical plot depicting data relating to an embodiment of the invention.

FIG. 3 is a graphical plot depicting data relating to an embodiment of the invention.

FIG. 4 is a graphical plot depicting data relating to an embodiment of the invention.

FIG. 5 is a graphical plot depicting data relating to an embodiment of the invention.

FIG. 6 is a graphical plot depicting data relating to an embodiment of the invention.

FIG. 7 is a graphical plot depicting data relating to an embodiment of the invention.

FIG. 8 is a graphical plot depicting data relating to an embodiment of the invention.

FIG. 9 is a graphical plot depicting data relating to an embodiment of the invention.

FIG. 10 is a graphical plot depicting data relating to an embodiment of the invention.

FIG. 11 is a graphical plot depicting data relating to an embodiment of the invention.

FIG. 12 is a graphical plot depicting data relating to an embodiment of the invention.

FIG. 13 is a graphical plot depicting data relating to an embodiment of the invention.

FIG. 14 is a graphical plot depicting data relating to an embodiment of the invention.

FIG. 15 is a graphical plot depicting data relating to an embodiment of the invention.

FIG. 16 is a graphical plot depicting data relating to an embodiment of the invention.

FIG. 17 is a graphical plot depicting data relating to an embodiment of the invention.

FIG. 18 is a graphical plot depicting data relating to an embodiment of the invention.

FIG. 19 is a graphical plot depicting data relating to an embodiment of the invention.

FIG. 20 is a graphical plot depicting data relating to an embodiment of the invention.

FIG. 21 is a series of graphical plots depicting data relating to an embodiment of the invention.

FIG. 22 is a series of graphical plots depicting data relating to an embodiment of the invention.

FIG. 23 is a graphical plot depicting data relating to an embodiment of the invention.

FIG. 24 is a simplified block diagram illustrating a prior art extracorporeal filtration and detoxification system.

### DETAILED DESCRIPTION OF THE INVENTION

The present disclosure is based on the unexpected finding that certain cells are capable of responding to pro-inflammatory factors by secretion of specific anti-inflammatory factors. Such pro-inflammatory factors may be utilized to induce an anti-inflammatory response and/or inhibit an inflammatory response in a cell, thereby treating disease.

Before the present compositions and methods are further described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

The principles and operation of the methods according to the present disclosure may be better understood with reference to the figures and accompanying descriptions.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of skill in the art to which this disclosure belongs. Although any methods and materials similar or equivalent to those described herein can be used in the practice or testing of the disclosure, some preferred methods and materials are now described.

The invention described herein relates to a pro-inflammatory composition which includes one or more pro-inflammatory molecules, such as pro-inflammatory cytokines. The composition may be used to produce pharmaceutical compositions for use in treating a disease, disorder, or otherwise abnormal condition, such as an inflammatory disease or disorder.

As used herein, the term “subject” refers to a mammalian subject. As such, treatment of any animal in the order mammalian is envisioned. Such animals include, but are not limited to horses, cats, dogs, rabbits, mice, goats, sheep, non-human primates and humans. Thus, the method of the present disclosure is contemplated for use in veterinary applications as well as human use.

“Treatment” of a subject herein refers to both therapeutic treatment and prophylactic or preventative measures. Those in need of treatment include those already with a disease or disorder as well as those in which it is to be prevented. Hence, the subject may have been diagnosed as having a disease or disorder or may be predisposed or susceptible to a disease or disorder.

The expression “effective amount” refers to an amount of a pro-inflammatory molecule, such as a pro-inflammatory cytokine, that is effective for preventing, ameliorating or treating a disease or disorder. Such an effective amount will generally result in an improvement in the signs, symptoms or other indicators of a disease or disorder. For example, in liver diseases, an effective amount results in the reduction of biochemical markers indicative or poor hepatic function.

A “symptom” of a disease or disorder is any morbid phenomenon or departure from the normal in structure, function, or sensation, experienced by the subject and indicative of a disease or disorder.

As used herein, “inflammatory disease, disorder, or otherwise abnormal condition,” may include disorders associated with inflammation or have an inflammation component, such as, but are not limited to: sepsis, infection (such as viral, bacterial or fungal infection), acne vulgaris, asthma, chronic obstructive pulmonary disease (COPD), autoimmune diseases, celiac disease, chronic (plaque) prostatitis, glomerulonephritis, hypersensitivities, inflammatory bowel diseases (IBD, Crohn's disease, ulcerative colitis), pelvic inflammatory disease, reperfusion injury, rheumatoid arthritis, sarcoidosis, transplant rejection, vasculitis, interstitial cystitis, atherosclerosis, allergies (type 1, 2, and 3 hypersensitivity, hay fever), inflammatory myopathies, as systemic sclerosis, and include dermatomyositis, polymyositis, inclusion body myositis, Chediak-Higashi syndrome, chronic granulomatous disease, Vitamin A deficiency, cancer (solid tumor, gallbladder carcinoma), periodontitis, granulomatous inflammation (tuberculosis, leprosy, sarcoidosis, and syphilis), fibrinous inflammation, purulent inflammation, serous inflammation, ulcerative inflammation, and ischaemic heart disease, type I diabetes, and diabetic nephropathy.

In certain embodiments, the inflammatory disease, disorder, or otherwise abnormal condition includes many autoimmune diseases or disorders that are associated with inflammation or have an inflammation component, e.g., corresponding to one or more types of hypersensitivity. Exemplary autoimmune diseases or disorders that correspond to one or more types of hypersensitivity include: atopic allergy, atopic dermatitis, autoimmune hemolytic anemia, autoimmune hepatitis, autoimmune polyendocrine syndrome, autoimmune urticaria, celiac disease, cold agglutinin disease, contact dermatitis, Crohn's disease, diabetes mellitus type 1, discoid lupus erythematosus, Erythroblastosis fetalis, Goodpasture's syndrome, Graves' disease, Guillain-Barre syndrome (GBS), Hashimoto's encephalopathy, Hashimoto's thyroiditis, idiopathic thrombocytopenic purpura, autoimmune thrombocytopenic purpura, IgA nephropathy, lupus erythematosus, Meniere's disease, multiple sclerosis, myasthenia gravis, narcolepsy, neuromyelitis optica, Devic's disease, neuromyotonia, ocular cicatricial pemphigoid, opsoclonus myoclonus syndrome, PANDAS (pediatric autoimmune neuropsychiatric disorders associated with streptococcus), paraneoplastic cerebellar degeneration, pemphigus vulgaris, pernicious anaemia, psoriasis, psoriatic arthritis, rheumatoid arthritis, rheumatic fever, sarcoidosis, scleroderma, subacute bacterial endocarditis (SBE), systemic lupus erythematosis, Lupus erythematosis, temporal arteritis (also known as “giant cell arteritis”), thrombocytopenia, ulcerative colitis, undifferentiated connective tissue disease, urticarial vasculitis, and vasculitis.

Inflammatory disease, disorder, or otherwise abnormal condition in liver may include fatty liver disease, cirrhosis, liver cancer, and acute or chronic hepatitis caused by viral infection (e.g., by Hepatitis A, B, C, D and E), alcoholic hepatitis, drug or chemical intoxication (such as carbon-tetrachloride, amethopterin, tetracycline, acetaminophen, fenoprofen, and the like), mononucleosis, amebic dysentery, and other systematic infections by Epstein-Barr virus (EBV), cytomegalovirus (CMV), or bacteria.

Inflammatory disease, disorder, or otherwise abnormal condition in kidney may be associated with acute or chronic nephritis, interstitial nephritis, lupus nephritis, IgA nephropathy (Berger's disease), glomerulonephritis, membranoproliferative glomerulonephritis (MPGN), autoimmune disorders related to chronic kidney disease (CKD) and inflammation, Goodpasture's syndrome, Wegener's granulomatosis, pyelonephritis, athletic nephritis, kidney stones, and gout.

Inflammatory bowel disease (IBD) is a group of inflammatory conditions of the colon and small intestine. The major types of IBD are Crohn's disease and ulcerative colitis. Other forms of IBD, which are not always classified as typical IBD, include collagenous colitis, lymphocytic colitis, ischaemic colitis, diversion colitis, Behcet's disease, and indeterminate colitis.

Inflammatory disease, disorder, or otherwise abnormal condition in pancreas includes various forms of pancreatitis with a variety of causes and symptoms, including pancreatitis caused by alcohol, gallstone, medication (e.g., use of corticosteroids such as prednisolone, HIV drugs such as didanosine and pentamidine, diuretics, the anticonvulsant valproic acid, the chemotherapeutic agents L-asparaginase and azathioprine, estrogen by way of increased blood triglycerides, cholesterol-lowering statins, and the antihyperglycemic agents like metformin, vildagliptin, sitagliptin, and diabetes drug gliptins), trauma, mumps, autoimmune disease, scorpion stings, high blood calcium, high blood triglycerides, hypothermia, endoscopic retrograde cholangiopancreatography (ERCP), Pancreas divisum, pregnancy, diabetes mellitus type 2, pancreatic cancer, pancreatic duct stones, vasculitis (inflammation of the small blood vessels in the pancreas), coxsackievirus infection, and porphyria—particularly acute intermittent porphyria and erythropoietic protoporphyria, viral infection (by coxsackie virus, cytomegalovirus, Hepatitis B, herpes simplex virus, mumps, varicella-zoster virus), bacterial infection (Legionella, Leptospira, Mycoplasma, Salmonella), fungal infection (Aspergillus), or parasitic infection (Ascaris, Cryptosporidium, Toxoplasma).

As used herein, “associated with the inflammatory disease” refers to the situation that the pro-inflammatory cytokine is known to cause the inflammatory disease, disorder, or otherwise abnormal condition, exacerbates at least one symptom of the inflammatory disease, disorder, or otherwise abnormal condition, or is known to be overexpressed in the inflammatory disease, disorder, or otherwise abnormal condition.

The present invention provides a pro-inflammatory composition which includes one or more pro-inflammatory molecules. In embodiments, the pro-inflammatory molecule induces expression of one or more polypeptides by a cell contacted by the molecule. In one embodiment the pro-inflammatory molecule induces expression of one or more secretory or excreted factors by a cell contacted by the molecule. In one embodiment the pro-inflammatory molecule induces expression of one or more anti-inflammatory factors. In embodiments the pro-inflammatory molecule induces expression of one or more anti-inflammatory factors selected from Alpha-1-Antitrypsin (AAT), Interleukin-1 receptor antagonist (IL-1Ra), Interleukin-4 (IL-4), Interleukin-10 (IL-10), Interleukin-13 (IL-β), Interferon alpha (IFN-α), Gelsolin, Transforming Growth Factor beta (TGF-β), and any combination thereof.

In embodiments the pro-inflammatory molecule induces expression of one or more factors selected from Albumin, Alpha-1-Antitrypsin (AAT), Alpha-2-Macroglobulin (A2Macro), Alpha-Fetoprotein (AFP), Amphiregulin (AR), Angiopoeitin-2 (ANG-2), Apolipoprotein A-I (Apo A-I), Apolipoprotein A-II (Apo A-II), Apolipoprotein C-I (Apo C-I), Apolipoprotein C-III (Apo C-III), Apolipoprotein H (Apo H), Beta-2-Microglobulin (β2M), CD 40 antigen (CD40), Complement C3 (C3), CreatineKinase-MB (CK-MB), Eotaxin-1, Erythropoietin (EPO), soluble Fas receptor, Factor VII, Ferritin (FRTN), Fibrinogen, Gelsolin, Hepatocyte Growth Factor (HGF), Heparin Binding Epidermal Growth Factor (HB-EGF), Human Chorionic Gonadotropin beta (hCG), Intercellular Adhesion Molecule 1 (ICAM-1), Interleukin-1 receptor antagonist (IL-1Ra), Interleukin-8 (IL-8), Macrophage-Derived Chemokine (MDC), Neuron-Specific Enolase (NSE), Neutrophil Gelatinase-associated Lipocalin (NGAL), Placental Growth Factor (PlGF), Plasminogen Activator Inhibitor 1 (PAI-1), Platelet-derived Growth Factor BB (PDGF-BB), Serotransferrin (Transferrin), Sex Hormone-Binding Globulin (SHBG), Stem Cell Factor (SCF), T-Cell-Specific Protein RANTES (RANTES), Thyroxine-Binding Globulin (TBG), Tissue Inhibitor of Metalloproteinse 1 (TIMP-1), Transforming Growth Factor alpha (TGFα), Transthyretin (TTR), Vascular Endothelial Growth Factor (VEGF), Vascular Endothelial Growth Factor C (VEGF-C), soluble Fas, and any combination thereof.

In various embodiments, expression of the anti-inflammatory factor is increased by a factor of at least 2.0, 5.0, 10, 25, 50, 100, 250, 500, 1,000, 5,000 or greater as compared to expression prior to contacting with the pro-inflammatory molecule.

A pro-inflammatory molecule useful in the invention can be any type of molecule, for example, a polynucleotide, a peptide, a peptidomimetic, peptoids such as vinylogous peptoids, chemical compounds, such as organic molecules or small organic molecules, or the like.

In embodiments the pro-inflammatory composition of the disclosure includes one or more pro-inflammatory polypeptides, such as a pro-inflammatory cytokine. In embodiments, the composition is a pharmaceutical composition that includes one or more pro-inflammatory molecules, such as a polypeptide and a pharmaceutically acceptable carrier. The terms “polypeptide”, “peptide”, or “protein” are used interchangeably herein to designate a linear series of amino acid residues connected one to the other by peptide bonds between the alpha-amino and carboxy groups of adjacent residues.

In embodiments, the composition includes a single type of pro-inflammatory polypeptide. In other embodiments, the pharmaceutical composition includes a combination of two or more pro-inflammatory polypeptides, such as IL-6 and IL-1β. In embodiments, the composition is substantially free of blood proteins and/or metabolites found in the blood. In other embodiments, the composition includes serum albumin (e.g., human serum albumin). In embodiments, any polypeptide present in a composition is recombinantly produced. In embodiments, any polypeptide present in a composition is produced in-vivo by a cell in a subject.

In embodiments, the composition includes one or more pro-inflammatory polypeptides selected from TNF-α, Interleukin-1 (IL-1), Interleukin-5 Interleukin-6 (IL-6), Interleukin-8 (IL-8), Interleukin-11 (IL-11), Interleukin-12 (IL-12), interleukin-17 (IL-17), Interleukin-18 (IL-18), Interleukin-1 beta (IL-1β), Monocyte chemotactic protein-1 (MCP-1), Macrophage inflammatory protein 1-alpha (MIP-1α), Macrophage inflammatory protein 1-beta (MIP-1β), Interleukin-8 (IL-8), Interferon gamma (IFN-γ), Granulocyte-macrophage colony-stimulating factor (GM-CSF), lymphotactin, fractalkine, or any combination thereof.

Toll-like receptors (TLRs) recognize pathogen-associated molecular patterns (PAMPs) to detect the presence of pathogens. TLRs are expressed on both immune cells, Kupffer cells, endothelial cells, dendritic cells, biliary epithelial cells, hepatic stellate cells, and hepatocytes. TLR signaling induces potent innate immune responses in these and other cell types. As such the composition may include one or more PAMPs.

TLRs also play a role in the regulation of inflammation based on their ability to recognize endogenous TLR ligands termed damage-associated molecular patterns (DAMPs). As such the composition may include one or more DAMPs.

In embodiments the composition may include a toll-like receptor or a NOD-like receptor which functions as an immunostimulant. The toll-like receptor may include a member selected from TLR-1, TLR-2, TLR-3, TLR-4, TLR-5, TLR-6, TLR-7, TLR-8 and TLR-9, but not be limited thereto. The NOD-like receptor may include, for example, NLRA, NLRB, NLRC or NLRP, but not be limited thereto.

In one embodiment, the one or more pro-inflammatory molecules includes LPS, poly(I:CU), CpG, imiquimod, resiquimod, dSLIM, MPLA, flagellin, a plasmid DNA double-strand DNA, a single-strand DNA, a saponin, or any combination thereof.

In embodiments, the pro-inflammatory molecule is a polynucleotide, such as an antisense oligonucleotide or RNA molecule which increases expression and/or activity (directly or indirectly) of an anti-inflammatory factor. In various aspects, the pro-inflammatory molecule may be a polynucleotide, such as an antisense oligonucleotide or RNA molecule, such as microRNA, dsRNA, siRNA, stRNA, and shRNA.

MicroRNAs (miRNA) are single-stranded RNA molecules, which regulate gene expression. miRNAs are encoded by genes from whose DNA they are transcribed but miRNAs are not translated into protein; instead each primary transcript (a pri-miRNA) is processed into a short stem-loop structure called a pre-miRNA and finally into a functional miRNA. Mature miRNA molecules are either fully or partially complementary to one or more messenger RNA (mRNA) molecules, and their main function is to down-regulate gene expression. MicroRNAs can be encoded by independent genes, but also be processed (via the enzyme Dicer) from a variety of different RNA species, including introns, 3′ UTRs of mRNAs, long noncoding RNAs, snoRNAs and transposons. As used herein, microRNAs also include “mimic” microRNAs which are intended to mean a microRNA exogenously introduced into a cell that have the same or substantially the same function as their endogenous counterpart. Thus, while one of skill in the art would understand that an agent may be an exogenously introduced RNA, an agent also includes a compound or the like that increase or decrease expression of microRNA in the cell.

The terms “small interfering RNA” and “siRNA” also are used herein to refer to short interfering RNA or silencing RNA, which are a class of short double-stranded RNA molecules that play a variety of biological roles. Most notably, siRNA is involved in the RNA interference (RNAi) pathway where the siRNA interferes with the expression of a specific gene. In addition to their role in the RNAi pathway, siRNAs also act in RNAi-related pathways (e.g., as an antiviral mechanism or in shaping the chromatin structure of a genome).

The term “polynucleotide” or “nucleotide sequence” or “nucleic acid molecule” is used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the terms as used herein include naturally occurring nucleic acid molecules, which can be isolated from a cell, as well as synthetic polynucleotides, which can be prepared, for example, by methods of chemical synthesis or by enzymatic methods such as by the polymerase chain reaction (PCR). It should be recognized that the different terms are used only for convenience of discussion so as to distinguish, for example, different components of a composition.

As discussed herein, the composition of the disclosure can include a single pro-inflammatory polypeptide, or combinations thereof. The composition can be substantially free of proteins and other polypeptides that are anti-inflammatory. The composition can be substantially free of any anti-inflammatory molecules. As used herein, the term “substantially free of proteins and other polypeptides” means that less than 5% of the protein content of the composition is made up of proteins and other polypeptides that are not a pro-inflammatory polypeptide. As used herein, the term “substantially free of an anti-inflammatory molecule” means that less than 5% of the content of the composition is made up of an anti-inflammatory molecule. A composition that is substantially free of non-pro-inflammatory polypeptides can have less than 4%, 3%, 2%, 1%, 0.5%, 0.1%, 0.05%, 0.01%, or less of proteins or other polypeptides that are anti-inflammatory. A composition that is substantially free of an anti-inflammatory molecule can have less than 4%, 3%, 2%, 1%, 0.5%, 0.1%, 0.05%, 0.01%, or less of such molecules. Thus, the composition can be substantially free of blood proteins, such as serum albumin, globulins, fibrinogen, and clotting factors. Alternatively, the composition can include one or more of serum albumin, globulins, fibrinogen, and clotting factors.

In embodiments, the pro-inflammatory polypeptide is not naturally found in a human or other mammal or animal. For example, the polypeptide may be synthetic, recombinant or the like. However, a composition of the invention can include a pro-inflammatory polypeptide that is naturally found in a human or other mammal or animal.

In embodiments, the pro-inflammatory polypeptide may include a non-naturally occurring amino acid. “Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, .gamma.-carboxyglutamate, and O-phosphoserine. “Amino acid analogs” refers to compounds that have the same fundamental chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

In embodiments, the composition includes one or more conservatively modified variants of a pro-inflammatory polypeptide of the present invention. In embodiments, the conservatively modified variant has at least 80% sequence similarity, often at least 85% sequence similarity, 90% sequence similarity, or at least 95%, 96%, 97%, 98%, or 99% sequence similarity at the amino acid level, with the naturally occurring polypeptide.

With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another: 1) Alanine (A), Glycine (G): 2) Aspartic acid (D), Glutamic acid (E), 3) Asparagine (N), Glutamine (Q), 4) Arginine (R), Lysine (K), 5) Isoleucine (I), Leucine (L), Methionine (M), Valine (V), 6) Phenylalanine (F), Tyrosine (Y), Tryptophan (W), 7) Serine (S), Threonine (T), and 8) Cysteine (C), Methionine (M) (see, e.g., Creighton, Proteins (1984)).

The terms “identical” or percent “identity,” in the context of two or more polypeptide sequences, refer to two or more sequences or subsequences that are the same or have a specified percentage of amino acid residues that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithm with default parameters, or by manual alignment and visual inspection. Such sequences are then said to be “substantially identical.”

In embodiments, the composition is substantially free of biological molecules (such as non-pro-inflammatory polypeptides, nucleic acids, lipids, carbohydrates, and metabolites) that are associated with the pro-inflammatory polypeptide in vivo or co-purify with the pro-inflammatory polypeptide. As used herein, the term “substantially free of biological molecules” means that less than 5% of the dry weight of the composition is made up of biological molecules that are not pro-inflammatory polypeptides. A composition that is substantially free of such biological molecules can have less than 4%, 3%, 2%, 1%, 0.5%, 0.1%, 0.05%, 0.01%, or less of biological molecules that are not pro-inflammatory polypeptides. Thus, for example, the composition can be substantially free of biological molecules that are abundant in the blood, such as, fatty acids, cholesterol, non-protein clotting factors, metabolites, and the like. In addition, the composition can be substantially free of cells, including red blood cells, white blood cells, platelets, and cell fragments.

In embodiments, the composition of the invention includes at least 1 mg (e.g., at least 5, 10, 20, 30, 40, 50, 75, 100, 150, 200, 250, 300, 400, 500, 600, 700, 800, 900, 1000 mg, or more) of pro-inflammatory molecule. Thus, for example, the compositions can include an amount of pro-inflammatory molecule equal to about 1 mg to about 1000 mg (e.g., about 5 mg to about 900 mg, about 5 mg to about 800 mg, about 5 mg to about 700 mg, about 5 mg to about 600 mg, about 10 mg to about 500 mg, about 10 mg to about 400 mg, about 10 mg to about 300 mg, about 10 mg to about 250 mg, about 10 mg to about 200 mg, about 10 mg to about 150 mg, about 10 mg to about 100 mg, about 50 mg to about 500 mg, about 50 mg to about 400 mg, about 50 mg to about 300 mg, about 50 mg to about 250 mg, about 50 mg to about 200 mg, about 50 mg to about 150 mg, about 50 mg to about 100 mg, about 75 mg to about 500 mg, about 75 mg to about 400 mg, about 75 mg to about 300 mg, about 75 mg to about 250 mg, about 75 mg to about 200 mg, about 75 mg to about 150 mg, about 75 mg to about 100 mg, about 100 mg to about 500 mg, about 100 mg to about 400 mg, about 100 mg to about 300 mg, about 100 mg to about 250 mg, about 100 mg to about 200 mg, or any other range containing two of the foregoing endpoints).

In embodiments, the composition of the invention can include a solution that contains at least 1 mg/ml (e.g., at least 5, 10, 15, 20, 25, 30, 35, 40, 45, 50, 55, 60, 65, 70, 75, 80, 85, 90, 95, 100 mg/ml or more) of a pro-inflammatory molecule. Thus, for example, the composition can include a solution having a pro-inflammatory molecule concentration of about 1 mg/ml to about 1000 mg/ml (e.g., about 5 mg/ml to about 900 mg/ml, about 5 mg/ml to about 800 mg/ml, about 5 mg/ml to about 700 mg/ml, about 5 mg/ml to about 600 mg/ml, about 5 mg/ml to about 500 mg/ml, about 10 mg/ml to about 500 mg/ml, about 10 mg/ml to about 400 mg/ml, about 10 mg/ml to about 300 mg/ml, about 10 mg/ml to about 250 mg/ml, about 10 mg/ml to about 200 mg/ml, about 10 mg/ml to about 150 mg/ml, about 10 mg/ml to about 100 mg/ml, about 50 mg/ml to about 500 mg/ml, about 50 mg/ml to about 400 mg/ml, about 50 mg/ml to about 300 mg/ml, about 50 mg/ml to about 250 mg/ml, about 50 mg/ml to about 200 mg/ml, about 50 mg/ml to about 150 mg/ml, about 50 mg/ml to about 100 mg/ml, about 75 mg/ml to about 500 mg/ml, about 75 mg/ml to about 400 mg/ml, about 75 mg/ml to about 300 mg/ml, about 75 mg/ml to about 250 mg/ml, about 75 mg/ml to about 200 mg/ml, about 75 mg/ml to about 150 mg/ml, about 75 mg/ml to about 100 mg/ml, about 100 mg/ml to about 500 mg/ml, about 100 mg/ml to about 400 mg/ml, about 100 mg/ml to about 300 mg/ml, about 100 mg/ml to about 250 mg/ml, about 100 mg/ml to about 200 mg/ml, about 10 mg/ml to about 150 mg/ml, or any other range containing two of the foregoing endpoints).

In embodiments, the composition of the invention includes at least 1 pg (e.g., at least 5, 10, 20, 30, 40, 50, 75, 100, 150, 200, 250, 300, 400, 500, 600, 700, 800, 900, 1000 pg, or more) of pro-inflammatory molecule. Thus, for example, the compositions can include an amount of pro-inflammatory molecule equal to about 1 pg to about 1000 pg (e.g., about 5 pg to about 900 pg, about 5 pg to about 800 pg, about 5 pg to about 700 pg, about 5 pg to about 600 pg, about 10 pg to about 500 pg, about 10 pg to about 400 pg, about 10 pg to about 300 pg, about 10 pg to about 250 pg, about 10 pg to about 200 pg, about 10 pg to about 150 pg, about 10 pg to about 100 pg, about 50 pg to about 500 pg, about 50 pg to about 400 pg, about 50 pg to about 300 pg, about 50 pg to about 250 pg, about 50 pg to about 200 pg, about 50 pg to about 150 pg, about 50 pg to about 100 pg, about 75 pg to about 500 pg, about 75 pg to about 400 pg, about 75 pg to about 300 pg, about 75 pg to about 250 pg, about 75 pg to about 200 pg, about 75 pg to about 150 pg, about 75 pg to about 100 pg, about 100 pg to about 500 pg, about 100 pg to about 400 pg, about 100 pg to about 300 pg, about 100 pg to about 250 pg, about 100 pg to about 200 pg, or any other range containing two of the foregoing endpoints).

In embodiments, the composition of the invention can include a solution that contains at least 1 pg/ml (e.g., at least 5, 10, 15, 20, 25, 30, 35, 40, 45, 50, 55, 60, 65, 70, 75, 80, 85, 90, 95, 100 pg/ml or more) of a pro-inflammatory molecule. Thus, for example, the composition can include a solution having a pro-inflammatory molecule concentration of about 1 pg/ml to about 1000 pg/ml (e.g., about 5 pg/ml to about 900 pg/ml, about 5 pg/ml to about 800 pg/ml, about 5 pg/ml to about 700 pg/ml, about 5 pg/ml to about 600 pg/ml, about 5 pg/ml to about 500 pg/ml, about 10 pg/ml to about 500 pg/ml, about 10 pg/ml to about 400 pg/ml, about 10 pg/ml to about 300 pg/ml, about 10 pg/ml to about 250 pg/ml, about 10 pg/ml to about 200 pg/ml, about 10 pg/ml to about 150 pg/ml, about 10 pg/ml to about 100 pg/ml, about 50 pg/ml to about 500 pg/ml, about 50 pg/ml to about 400 pg/ml, about 50 pg/ml to about 300 pg/ml, about 50 pg/ml to about 250 pg/ml, about 50 pg/ml to about 200 pg/ml, about 50 pg/ml to about 150 pg/ml, about 50 pg/ml to about 100 pg/ml, about 75 pg/ml to about 500 pg/ml, about 75 pg/ml to about 400 pg/ml, about 75 pg/ml to about 300 pg/ml, about 75 pg/ml to about 250 pg/ml, about 75 pg/ml to about 200 pg/ml, about 75 pg/ml to about 150 pg/ml, about 75 pg/ml to about 100 pg/ml, about 100 pg/ml to about 500 pg/ml, about 100 pg/ml to about 400 pg/ml, about 100 pg/ml to about 300 pg/ml, about 100 pg/ml to about 250 pg/ml, about 100 pg/ml to about 200 pg/ml, about 10 pg/ml to about 150 pg/ml, or any other range containing two of the foregoing endpoints).

The composition of the invention is typically a pharmaceutical composition. Such a pharmaceutical composition can include one or more pro-inflammatory molecules and a pharmaceutically acceptable carrier. A pharmaceutical composition can further include a protein other than a pro-inflammatory molecule of the invention. The other protein can be a therapeutic agent, such as a therapeutic polypeptide. Alternatively, the other protein can be a carrier protein, such as serum albumin (e.g., HSA). By mixing the pro-inflammatory molecule(s) in the pharmaceutical composition with serum album, the pro-inflammatory molecules can be effectively “loaded” onto the serum albumin.

In embodiments, the composition of the invention includes an anti-coagulant, such as heparin or citrate. As used herein, “citrate” refers to a citrate anion, in any form, including citric acid (citrate anion complexed with three protons), salts containing citrate anion, and partial casters of citrate anion. Citrate anion is an organic tricarboxylate. Citric acid, which has been assigned Chemical Abstracts Registry No. 77-92-2, has the molecular formula HOC(CO₂H)(CH₂CO₂H)₂ and a formula weight of 192.12 g/mol. A citrate salt (i.e., a salt containing citrate anion) is composed of one or more citrate anions in association with one or more physiologically-acceptable cations. Exemplary physiologically-acceptable cations include, but are not limited to, protons, ammonium cations and metal cations. Suitable metal cations include, but are not limited to, sodium, potassium, calcium, and magnesium, where sodium and potassium are preferred, and sodium is more preferred. A composition containing citrate anion may contain a mixture of physiologically-acceptable cations.

In one embodiment, the composition includes sodium citrate. Sodium citrate may be in the form of a dry chemical powder, crystal, pellet or tablet. Any physiologically tolerable form of citric acid or sodium citrate may be used. For instance, the citric acid or sodium citrate may be in the form of a hydrate, including a monohydrate.

The pharmaceutical composition of the invention may be prepared by mixing one or more pro-inflammatory molecules having the desired degree of purity with optional pharmaceutically acceptable carriers, excipients or stabilizers (Remington's Pharmaceutical Sciences, 16th edition, Osol, A. Ed. (1980)). Acceptable carriers, excipients, or stabilizers are nontoxic to recipients at the dosages and concentrations employed, and may include buffers such as phosphate, citrate, and other organic acids; antioxidants including ascorbic acid and methionine, preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol, 3-pentanol, and m-cresol); low molecular weight (less than about 10 residues) polypeptides, proteins, such as serum albumin, gelatin, or immunoglobulins; hydrophilic polymers such as polyvinylpyrrolidone amino acids such as glycine, glutamine, asparagine, histidine, arginine, or lysine, monosaccharides, disaccharides, and other carbohydrates including glucose, mannose, or dextrins, chelating agents such as EDTA, sugars such as sucrose, mannitol, trehalose or sorbitol, salt-forming counter-ions such as sodium, metal complexes (for example, Zn-protein complexes) and/or non-ionic surfactants such as TWEEN™, PLURONICS™ or polyethylene glycol (PEG).

In embodiments, the composition of the present invention may include live cells. In one embodiment, the composition includes a hepatocyte cell. In one embodiment, the composition includes HepG2 cells or C3A cells which are optionally recombinantly engineered.

The pro-inflammatory molecules of the invention provide powerful tools for inducing an anti-inflammatory response in a cell and/or treating a disease or disorder, such as an inflammatory disease.

Accordingly, the invention provides a method of inducing an anti-inflammatory response, or inhibiting an inflammatory response in a cell by increasing the expression level of at least one (e.g., 2, 3, 4, 5, or more) anti-inflammatory peptide in the cell. The method includes contacting a cell with a pro-inflammatory molecule of the invention. In embodiments the pro-inflammatory molecule induces expression of one or more anti-inflammatory factors selected from AAT, IL-1Ra, IL-4, IL-10, IL-13, IFN-α, gelsolin, TGF-β and any combination thereof. In embodiments the pro-inflammatory molecule induces expression of one or more factors selected from Albumin, Alpha-1-Antitrypsin (AAT), Alpha-2-Macroglobulin (A2Macro), Alpha-Fetoprotein (AFP), Amphiregulin (AR), Angiopoeitin-2 (ANG-2), Apolipoprotein A-I (Apo A-I), Apolipoprotein A-II (Apo A-II), Apolipoprotein C-I (Apo C-I), Apolipoprotein C-III (Apo C-III), Apolipoprotein H (Apo H), Beta-2-Microglobulin (β2M), CD 40 antigen (CD40), Complement C3 (C3), CreatineKinase-MB (CK-MB), Eotaxin-1, Erythropoietin (EPO), soluble Fas receptor, Factor VII, Ferritin (FRTN), Fibrinogen, Gelsolin, Hepatocyte Growth Factor (HGF), Heparin Binding Epidermal Growth Factor (HB-EGF), Human Chorionic Gonadotropin beta (hCG), Intercellular Adhesion Molecule 1 (ICAM-1), Interleukin-1 receptor antagonist (IL-1Ra), Interleukin-8 (IL-8), Macrophage-Derived Chemokine (MDC), Neuron-Specific Enolase (NSE), Neutrophil Gelatinase-associated Lipocalin (NGAL), Placental Growth Factor (PIGF), Plasminogen Activator Inhibitor 1 (PAI-1), Platelet-derived Growth Factor BB (PDGF-BB), Serotransferrin (Transferrin), Sex Hormone-Binding Globulin (SHBG), Stem Cell Factor (SCF), T-Cell-Specific Protein RANTES (RANTES), Thyroxine-Binding Globulin (TBG), Tissue Inhibitor of Metalloproteinse 1 (TIMP-1), Transforming Growth Factor alpha (TGFα), Transthyretin (TTR), Vascular Endothelial Growth Factor (VEGF), Vascular Endothelial Growth Factor C (VEGF-C), soluble Fas, and any combination thereof.

In various embodiments, expression of the anti-inflammatory factor is increased by a factor of at least 2.0, 5.0, 10, 25, 50, 100, 250, 500, 1,000, 5,000, 10,000, 50,000 or greater as compared to expression prior to contacting with the pro-inflammatory molecule.

The invention also provides a method of treating a disease or disorder in a subject. The method includes administering a pro-inflammatory molecule of the invention (or, for example, a pharmaceutical composition comprising a pro-inflammatory polypeptide) to the subject, or cell or tissue thereof.

In the method of the invention, the pro-inflammatory molecule induces expression of one or more anti-inflammatory factors by a contacted cell. In embodiments, the contacted cell is a eukaryotic cell, such as a mammalian cell. In one embodiment the contacted cell is a hepatocyte. In one embodiment, the cell is a hepatoblastoma-derived cell. In one embodiment, the cell is a HepG2 cell or a C3A cell of a C3A cell line. In one embodiment, the cell is a clonal derivative from a parental C3A cell line. In one embodiment, the cell is a recombinantly engineered cell.

The term “C3A cell line” refers to a sub-clone of the human hepatoblastoma cell line HepG2. The C3A cell line is a qualified cell line having been deposited at the American Type Culture Collection under ATCC No. CRL-10741.

Administration of the composition may be performed in any suitable manner including, for example, intravenously, intraperitoneally, parenteral, orthotopically, subcutaneously, topically, nasally, orally, sublingually, intraocularly, by means of an implantable depot, using nanoparticle-based delivery systems, microneedle patch, microspheres, beads, osmotic or mechanical pumps, and/or other mechanical means.

In various embodiments, a cell may be contacted by the composition in-vivo or in-vitro. In one embodiment, the cell is contacted ex-vivo, the cell being contained within an active cartridge (bioreactor) including live cells, such as the active cartridge of the extracorporeal detoxification system described in U.S. Pat. No. 8,105,491, which is incorporated herein by reference in its entirety. The system may be fluidly coupled to a subject, or a cell or organ thereof, e.g., a liver.

As indicated in FIG. 24, the extracorporeal detoxification system 10 generally includes a blood circuit 100 configured to be coupled to a patient and operative to communicate blood from the patient, through an ultrafiltrate generator (UFG) 40, and back to the patient; a recirculation circuit 50 coupled to the UFG 40 and operative to draw ultrafiltrate from the UFG 40 and to treat ultrafiltrate independently of cellular components of the blood; and a conduit junction 15 operative to recombine the ultrafiltrate in the recirculation circuit 50 and the cellular components in the blood circuit 100 prior to reintroduction to the patient. Also shown in FIG. 24 is an active cartridge 70 and oxygenator 60 arranged within the recirculation circuit 50. The active cartridge 70 is utilized to treat the ultrafiltrate.

The term “active cartridge” refers to a hollow fiber based cartridge comprising cells (such as, for example, cells of the C3A cell line) having utility in therapeutic applications and detoxification processes.

The term “blood circuit” refers to a circuit of tubing connected to a double lumen catheter and operative to circulate blood from a patient to a blood control unit and back to the patient.

The term “C3A cell line” refers to a sub-clone of the human hepatoblastoma cell line HepG2. In embodiments, C3A cells are contained in the extracapillary space of one or more active cartridges. The C3A cell line has been deposited at the American Type Culture Collection under ATCC No. CRL-10741.

The term “detoxification device” refers to a cartridge, canister, or other device that provides a means of removal of specific or non-specific molecules from a fluid stream. Examples would be a dialysis cartridge, an adsorption cartridge, or a filter.

The term “extracapillary space” (ECS) refers to space outside the hollow fibers of active cartridges or an ultrafiltrate generator. The ECS of active cartridges may generally house the C3A cells.

The term “intracapillary space” (ICS) refers to space inside the hollow fibers of active cartridges or an ultrafiltrate generator. The ICS is the flow path for whole blood or the ultrafiltrate fluid.

The term “recirculation circuit” refers to a circuit generally enabling filtration, detoxification, and treatment of ultrafiltrate fluid; in some implementations, a recirculation circuit generally encompasses a reservoir, an oxygenator, and one or more active cartridges.

The term “ultrafiltrate” (UF) refers to plasma fluid and dissolved macromolecules filtered across the semi-permeable membrane of an ultrafiltrate generator.

The term “ultrafiltrate generator” (UFG) refers to a device comprising or embodied as a “blank” active cartridge (i.e., a hollow fiber cartridge which does not contain therapeutically active cells) and operative to separate plasma fluid (ultrafiltrate) from cellular blood components. The hollow fibers may be composed of a semi-permeable membrane which has, for example, a nominal molecular weight cut-off of approximately 100,000 Daltons in some implementations. During use of the UFG, blood may be circulated through the ICS of the hollow fibers; ultrafiltrate, comprising blood plasma and various macromolecules, passes through the membrane fiber walls into the recirculation circuit, where it is circulated through one or more active cartridges.

The term “ultrafiltration” refers generally to a process during which ultrafiltrate is pulled from whole blood across the semi-permeable membrane of the UFG. In some embodiments described below, an ultrafiltrate pump may control the rate of ultrafiltrate production, while the pore size of the hollow fiber membrane of the UFG may control the amount of ultrafiltrate permeating the membrane.

During clinical or therapeutic treatment, UF may be pumped through the lumen (ICS) of the hollow fiber cartridge within the active cartridge 70, allowing toxins, nutrients, glucose, and dissolved oxygen from the UF to diffuse across the membrane into the ECS, where the live cells may metabolize them. Metabolites, along with albumin and other proteins produced by the cells, may diffuse back across the membrane into the UF for return to the patient.

As set forth above and contemplated herein, the C3A cell line is a subclone of the human hepatoblastoma cell line HepG2. Some subclones of this parent cell line, such as C3A, for example, exhibit liver-specific functional capabilities such as high albumin production and α-fetoprotein (AFP) production as well as expression of anti-inflammatory mediator proteins α-1-antitrypsin (AAT) and IL-1Ra in response to pro-inflammatory molecules of the present invention, including for example, cytokines IL-6 and IL-1β.

In various embodiments, the system may be fluidly coupled to the subject, or a cell or organ thereof, e.g., a liver. The composition of the present invention is introduced into the blood circuit of system 10. The composition may be introduced into the circulatory of the subject, or introduced directly into the blood flow path of the system. In one embodiment, the composition is generated by cells of the subject being treated and flows into the blood circuit of system 10 during treatment.

Once in the blood circuit 100 of system 10, the pro-inflammatory molecules of the composition of the invention contact cells within the active cartridge thereby inducing expression and secretion of anti-inflammatory factors into UF. The UF is reintroduced into the blood flow path and reintroduced into the subject wherein the anti-inflammatory factors produced by the C3A cells contact cells of the subject, such as liver cells, thereby facilitating treatment of a disease or disorder.

While the cells of the active cartridge are illustrated as being C3A cells in the present embodiment, one of skill in the art would understand that the active cartridge could include any number of suitable cell types which are beneficial in treating any number of different diseases, such as inflammatory diseases as disclosed herein. In embodiments, the active cartridge may include cells recombinantly engineered to produce specific factors in response to molecules in the subject's blood.

In conjunction with any of the foregoing methods, the composition can be administered daily (or every other day, or weekly), wherein the amount of pro-inflammatory molecule is between about 1 mg and about 1000 mg (e.g., about 5 mg to about 900 mg, about 5 mg to about 800 mg, about 5 mg to about 700 mg, about 5 mg to about 600 mg, about 10 mg to about 500 mg, about 10 mg to about 400 mg, about 10 mg to about 300 mg, about 10 mg to about 250 mg, about 10 mg to about 200 mg, about 10 mg to about 150 mg, about 10 mg to about 100 mg, about 50 mg to about 500 mg, about 50 mg to about 400 mg, about 50 mg to about 300 mg, about 50 mg to about 250 mg, about 50 mg to about 200 mg, about 50 mg to about 150 mg, about 50 mg to about 100 mg, about 75 mg to about 500 mg, about 75 mg to about 400 mg, about 75 mg to about 300 mg, about 75 mg to about 250 mg, about 75 mg to about 200 mg, about 75 mg to about 150 mg, about 75 mg to about 100 mg, about 100 mg to about 500 mg, about 100 mg to about 400 mg, about 100 mg to about 300 mg, about 100 mg to about 250 mg, about 100 mg to about 200 mg, or any other range containing two of the foregoing endpoints).

In conjunction with any of the foregoing methods, the composition can be administered daily (or every other day, or weekly), wherein the amount of pro-inflammatory molecule is between about 1 pg and about 1000 pg (e.g., about 5 pg to about 900 pg, about 5 pg to about 800 pg, about 5 pg to about 700 pg, about 5 pg to about 600 pg, about 10 pg to about 500 pg, about 10 pg to about 400 pg, about 10 pg to about 300 pg, about 10 pg to about 250 pg, about 10 pg to about 200 pg, about 10 pg to about 150 pg, about 10 pg to about 100 pg, about 50 pg to about 500 pg, about 50 pg to about 400 pg, about 50 pg to about 300 pg, about 50 pg to about 250 pg, about 50 pg to about 200 pg, about 50 pg to about 150 pg, about 50 pg to about 100 pg, about 75 pg to about 500 pg, about 75 pg to about 400 pg, about 75 pg to about 300 pg, about 75 pg to about 250 pg, about 75 pg to about 200 pg, about 75 pg to about 150 pg, about 75 pg to about 100 pg, about 100 pg to about 500 pg, about 100 pg to about 400 pg, about 100 pg to about 300 pg, about 100 pg to about 250 pg, about 100 pg to about 200 pg, or any other range containing two of the foregoing endpoints).

In conjunction with any of the foregoing methods, the pro-inflammatory molecules (or pharmaceutical compositions comprising such molecules) can be administered in combination with a drug useful for treatment of the disease or disorder. In one embodiment, the composition is administered with an antibiotic. Examples of particular classes of antibiotics useful for synergistic therapy with the composition of the invention include aminoglycosides (e.g., tobramycin), penicillins (e.g., piperacillin), cephalosporins (e.g., ceftazidime), fluoroquinolones (e.g., ciprofloxacin), carbapenems (e.g., imipenem), tetracyclines and macrolides (e.g., erythromycin and clarithromycin). Further to the antibiotics listed above, typical antibiotics include aminoglycosides (amikacin, gentamicin, kanamycin, netilmicin, tobramycin, streptomycin, azithromycin, clarithromycin, erythromycin, erythromycin estolate/ethylsuccinate/gluceptate/lactobionate/stearate), beta-lactams such as penicillins (e.g., penicillin G, penicillin V, methicillin, nafcillin, oxacillin, cloxacillin, dicloxacillin, ampicillin, amoxicillin, ticarcillin, carbenicillin, mezlocillin, azlocillin and piperacillin), or cephalosporins (e.g., cephalothin, cefazolin, cefaclor, c-efamandole, cefoxitin, cefuroxime, cefonicid, cefmetazole, cefotetan, cefprozil, loracarbef, cefetamet, cefoperazone, cefotaxime, ceftizoxime, ceftriaxone, ceftazidime, cefepime, cefixime, cefpodoxime, and cefsulodin). Other classes of antibiotics include carbapenems (e.g., imipenem), monobactams (e.g., aztreonam), quinolones (e.g., fleroxacin, nalidixic acid, norfloxacin, ciprofloxacin, ofloxacin, enoxacin, lomefloxacin and cinoxacin), tetracyclines (e.g., doxycycline, minocycline, tetracycline), and glycopeptides (e.g., vancomycin, t-eicoplanin). Other antibiotics include chloramphenicol, clindamycin, trimethoprim, sulfa-methoxazole, nitrofurantoin, rifampin, mupirocin and the cationic peptides.

Any of the foregoing methods of the invention further include a step of assessing the efficacy of the therapeutic treatment. Because the pro-inflammatory molecules of the invention have a demonstrable ability to induce expression of anti-inflammatory factors, the efficacy of the therapeutic treatment can be assessed by measuring the levels of such factors (e.g., in the serum).

The following examples are provided to further illustrate the embodiments of the present invention, but are not intended to limit the scope of the invention. While they are typical of those that might be used, other procedures, methodologies, or techniques known to those skilled in the art may alternatively be used.

### Example 1

### Expression of Acute-Phase Proteins by C3A Cells

Background

Hepatitis caused by alcohol, drugs or toxins is characterized by elevated levels of IL-1, IL-6, and TNFα and an inflammatory cascade in which hepatocytes up-regulate acute-phase proteins (APP). Anti-TNFα or steroidal therapies have not demonstrated clinical benefit, thus, alternative cell-based strategies may be warranted.

Objectives

The purpose of this study was to evaluate the ability of qualified C3A cells of the invention to respond to selected inflammatory mediators found in ALD patients by producing factors associated with resolution of inflammation.

Materials and Methods

Conditioned media from C3A cells of an active cartridge of a treatment system of the disclosure were assayed for APP, cytokines, and other mediators that affect inflammation using singleplex ELISA kits (R&D Systems, abcam), chemiluminescent multiplex array kits (Aushon) and/or contracted services (Myriad Rules Based Medicine). Inflammatory cytokines reported to be present in hepatitis patient serum (IL-6 [0, 1, 10, 100 ng/mL]±IL-1β [0, 1, 10, 100 ng/mL]) were incubated with monolayer, or C3A cells recovered from the treatment system, for 24 or 54 h, and conditioned media were assayed for the APP IL-1 receptor antagonist (IL-1Ra), alpha-1-antitrypsin (AAT), and albumin. Lipopolysaccharide (LPS) (0, 0.01, 0.1, 1, 10 EU/mL) was incubated with monolayer C3A cells for 24 h and assayed for APP.

Results

When monolayer C3A cells were incubated with IL-6 only or IL-1β only, levels of IL-1Ra did not increase. However, co-incubation with both cytokines demonstrated a synergistic response towards increased IL-1Ra secretion at 24 h, which was further increased at 54 h (FIG. 1).

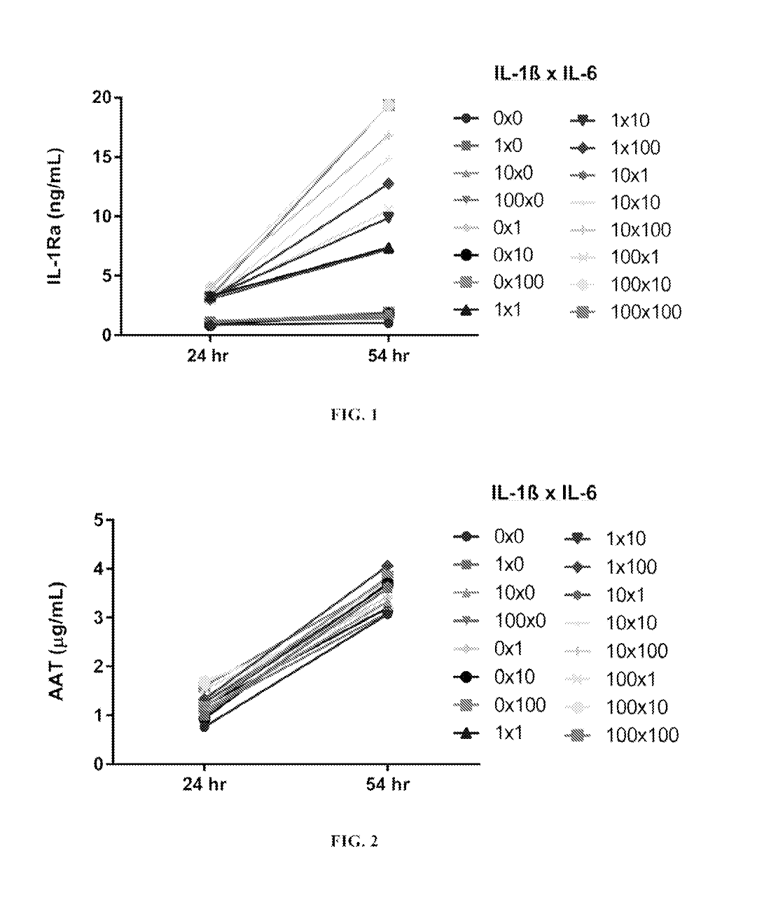

AAT, in general, was secreted at concentrations nearly 200-fold of IL-1Ra concentrations in C3A cells. AAT showed a more dose-dependent upregulation for both IL-6 and IL-1β at 24 h, and, although the values of AAT further increased at 54 h, the dose-dependency did not persist (FIG. 2).

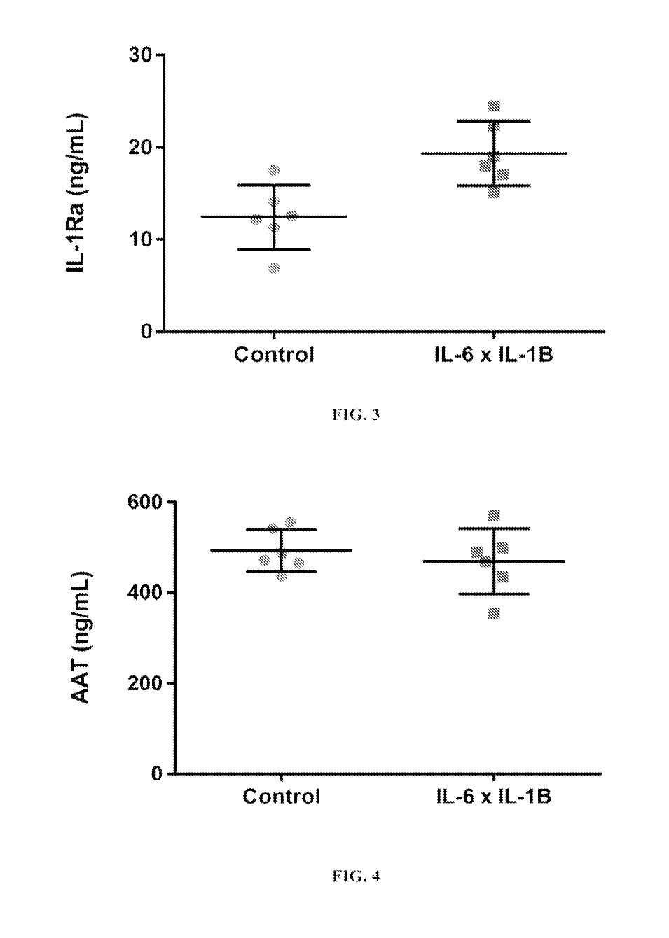

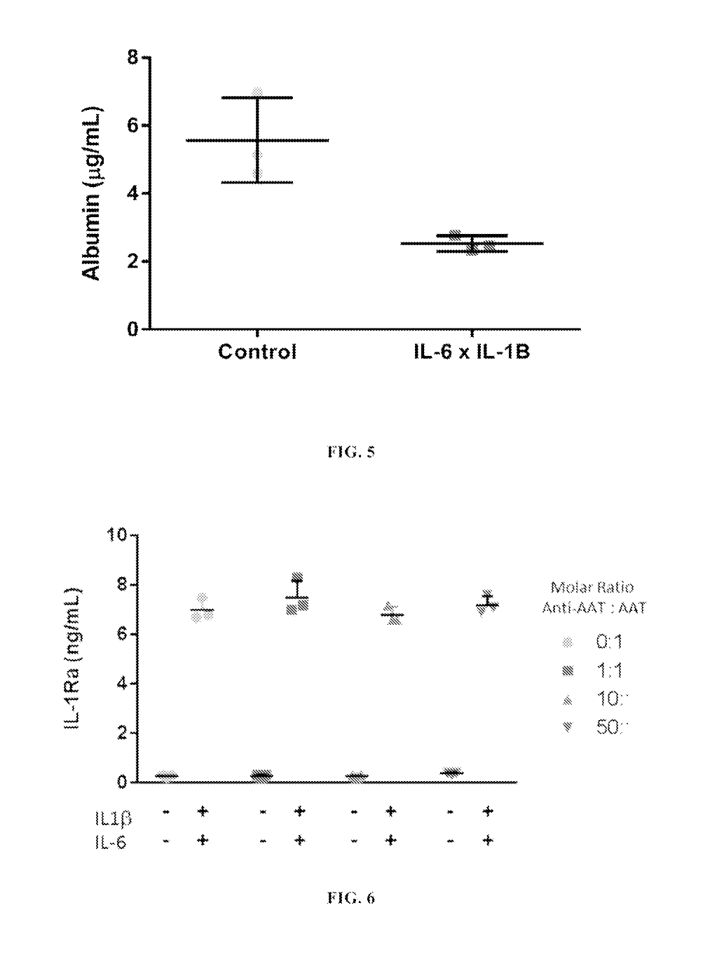

An increase in IL-1Ra (FIG. 3), but not in AAT (FIG. 4), was observed when tissue containing C3A cells was incubated with IL-6 and IL-1β (10 ng/mL each) for 24 h; however, the IL-1Ra response was not attenuated when neutralizing antibodies against AAT were present (FIG. 6). Albumin decreased in response to IL-6 and IL-1β (FIG. 5).

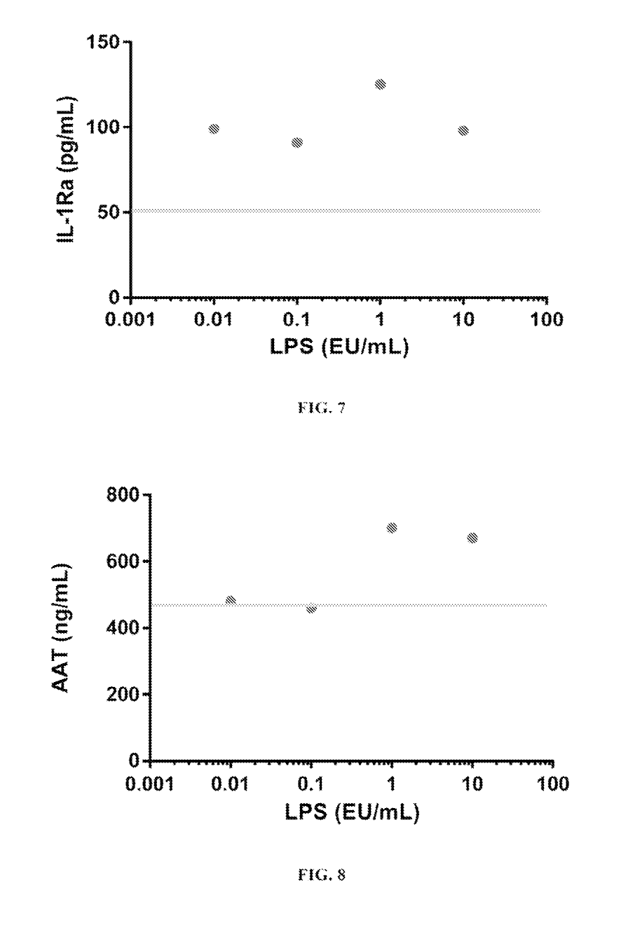

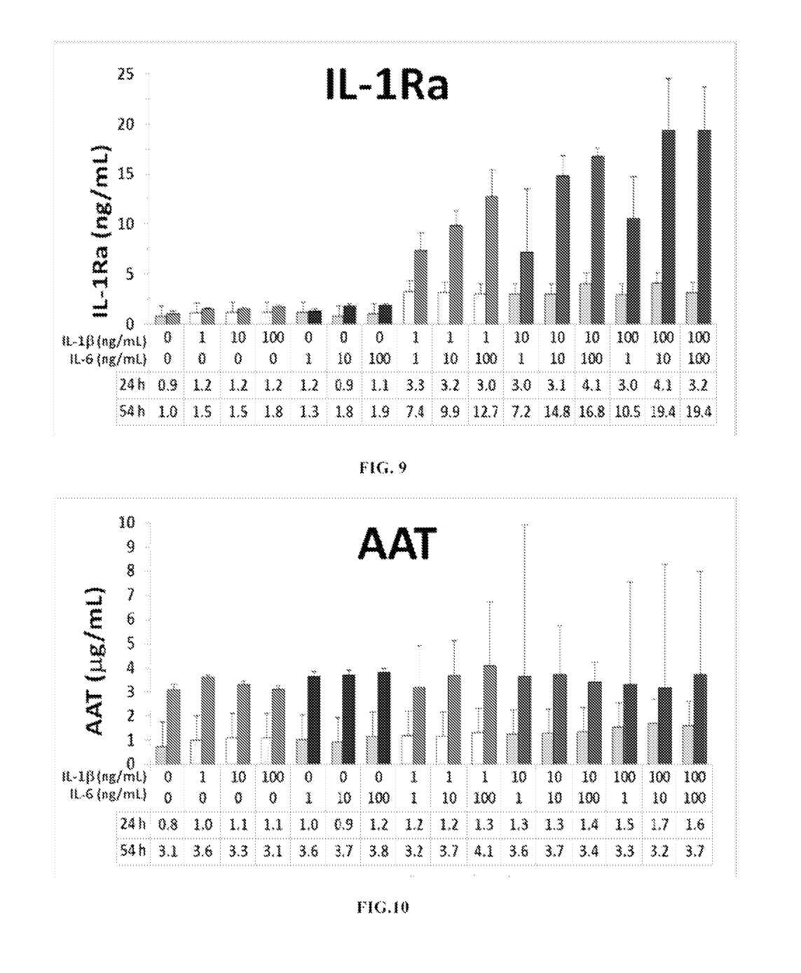

Direct exposure of monolayer C3A cells to LPS increased IL-1Ra at all concentrations evaluated (FIG. 7) and AAT levels only at higher concentrations (FIG. 8), but both were lower than observed with IL-6 and IL-1β stimulation.

Other factors that were observed to increase predominantly in IL-6-treated monolayer C3A cultures included fibrinogen (6-fold), IL-10 (10-fold), IL-18 (9-fold), MCP-1 (15-fold), and TNF-β (12-fold). Factors that were observed to increase predominantly in IL-1β-treated monolayer C3A cultures included G-CSF (50-fold), SCF (100-fold), IL-8 (2,000-fold), and TNFα (25-fold). Factors that were observed to increase predominantly when both IL-6 and IL-1β were present included ICAM-1 (9-fold), however, synergism between IL-6 and IL-11 was observed for G-CSF (300-fold).

Figure Legends

FIG. 1: Monolayer C3A cell secretion of IL-1Ra is upregulated in the combined presence of IL-6 and IL-1β and increases with exposure time. Samples are single replicates of pooled triplicate wells.

FIG. 2: Monolayer C3A cell secretion of AAT is upregulated in either IL-6 only, IL-1β only, or the combined presence of IL-6 and IL-1β and increases with exposure time. Samples are single replicates of pooled triplicate wells.

FIG. 3: C3A tissue secretion of IL-1Ra is also upregulated at 24 h in the combined presence of IL-6 (10 ng/mL) and IL-1β (10 ng/mL) (*p=0.0067). Results are mean±SD, n=6 replicates.

FIG. 4: C3A tissue secretion of AAT is not upregulated at 24 h in the combined presence of IL-6 (10 ng/mL) and IL-1β (10 ng/mL) (p=0.5140). Results are mean±SD, n=6 replicates.

FIG. 5: Monolayer C3A cell secretion of albumin is downregulated at 24 h in the combined presence of IL-6 (10 ng/mL) and IL-1β (10 ng/mL) (*p=0.04724) as expected for this APP. Results are mean±SD, n=3 replicates.

FIG. 6: Monolayer C3A cell secretion of IL-1Ra in response to the combined presence of IL-6 (10 ng/mL) and IL-1β (10 ng/mL) for 24 h. was not affected by treatment with a purported neutralizing anti-AAT antibody. Results are mean±SD, n=3 replicates.

FIG. 7: Monolayer C3A cell secretion of IL-1Ra is upregulated at 24 h in the presence of LPS. Samples are single replicates of pooled triplicate wells; pink line indicates untreated control response.

FIG. 8: Monolayer C3A cell secretion of AAT is upregulated at 24 h in the presence of higher concentrations of LPS. Samples are single replicates of pooled triplicate wells; pink line is untreated control.

Discussion

In the absence of stimulation, C3A cells in the active cartridge of the therapeutic system release several anti-inflammatory APP, but few pro-inflammatory cytokines or chemokines. Here, the ability of C3A cells to produce AAT and IL-1Ra in response to pro-inflammatory cytokines known to be associated with ALD-IL-1β and IL-6 is shown. Exogenous AAT and IL-1Ra have been shown to suppress pro-inflammatory cytokine synthesis by interference with TNFα and IL-1β pathways and enhancement of IL-10 production, the latter of which has broad anti-inflammatory properties. This effect has been reproduced in three different assay systems. The inability of the anti-AAT antibody to alter production of IL-1Ra suggests there is no autocrine effects of ATT on IL-1Ra in either positive or negative direction. Reduction of pro-inflammatory cytokines and increases in anti-inflammatory APP in response to elevated cytokines in ALD patients may contribute to resolution of inflammation by the ELAD System.

Conclusions

C3A cells have the ability to respond to inflammatory insult by secretion of anti-inflammatory mediators, IL-1Ra and AAT. This may represent one of the multiple mechanisms for the therapeutic benefit resolution in hepatitis patients treated with the therapeutic system of the disclosure.

### Example 2

### Expression of Acute-Phase Proteins by C3A Cells

Methods

Conditioned media from active cartridges including qualified C3A cells were assayed for APP, cytokines, and other mediators that affect inflammation. Inflammatory cytokines present in patient serum (IL-6±IL-1β) were incubated with monolayer, or C3A cells recovered from the system, for 24 or 54 h and media assayed for IL-1 receptor antagonist (IL-1Ra) and alpha-1-antitrypsin (AAT).

Results

When monolayer C3A cells were incubated with IL-6 or IL-1β, levels of IL-1Ra did not increase; however, co-incubation with both cytokines demonstrated a synergistic response towards increased IL-1Ra secretion at 24 h, which was further increased at 54 h. AAT showed a more dose-dependent up-regulation for both IL-6 and IL-1 β at 24 h, and, although the values of AAT further increased at 54 h, the dose-dependency did not persist. An increase in IL-1Ra, but not in AAT, was observed when ELAD C3A cells were incubated with IL-6 and IL-1β (10 ng/mL each) for 24 h. however, the IL-1Ra response was not attenuated when neutralizing antibodies against AAT were present.

Conclusions

C3A cells in the active cartridge of the therapeutic system release several anti-inflammatory APP, but few pro-inflammatory cytokines and chemokines which are considered pro-inflammatory. Exogenous AAT and IL-1Ra have been shown to suppress pro-inflammatory cytokine synthesis by interference with TNFα and IL-1β pathways and enhancement of IL-10 production, the latter of which has broad anti-inflammatory properties. Reduction of pro-inflammatory cytokines and increases in anti-inflammatory APP in response to elevated cytokines may contribute to resolution of inflammation by the ELAD System and may be part of the multiple mechanisms for the therapeutic benefit.

### Example 3

### Expression of Acute-Phase Proteins by C3A Cells

Background

A key pathogenesis of alcohol-induced liver decompensation (AILD) involves unregulated systemic inflammation. AILD patients have elevated plasma levels of inflammatory factors such as IL-1β, IL-6, lipopolysaccharide (LPS), and TNF-α. The diseased liver is unable to respond appropriately to these inflammatory mediators. This impaired immune response leaves the patient susceptible to systemic inflammatory syndrome which is associated with increased patient mortality.

In the healthy liver, hepatocytes are the major sources of acute-phase proteins (APPs) which contribute to control of systemic inflammation. Healthy hepatocytes respond to IL-1 and IL-6 (leading regulators of the APP response) by producing mediators of inflammatory resolution such as IL-1 receptor antagonist (IL-1Ra) and α1-antitrypsin (ATT). IL-1Ra and ATT have a demonstrated ability to mitigate systemic inflammation through competitive inhibition, protease inhibition, and blocking production of inflammatory signaling cascades.

Providing anti-inflammatory AAPs and other immune modulators (e.g. IL-10) to the AILD patient could provide therapeutic benefit.

Therapies, such as anti-TNF-α or steroid dosing have not demonstrated long-term clinical benefit. A multi-factor cell-based strategy utilizing the qualified C3A cell line of the invention along with the therapeutic system disclosed herein may be beneficial.

Objectives

The purpose of this study was to evaluate the ability of C3A cells of the invention to respond to selected inflammatory mediators (alone or in combination) commonly found in AILD patient plasma by secreting anti-inflammatory factors associated with the resolution of inflammation.

Materials and Methods

C3A cells of the invention were plated in monolayer and incubated with LPS or with inflammatory cytokines (IL-6, IL-1β, and/or TNF-α) for 24, 48, or 54 h. Cytokines were dosed individually or in combination at 0, 1, 10, or 100 ng/mL as indicated in each figure. Separate C3A cells were incubated with LPS at 0, 0.01, 0.1, 1, or 10 EU/mL for 24 h.

Additionally, intact C3A tissue (C3A cells cultured three-dimensionally between polysulfone hollow fibers) was excised from C3A cell cartridges and dosed in conjunction with 10 ng/mL IL-1β and 10 ng/mL IL-6 for 24 h. The supernatants from these monolayer C3A cell and ELAD C3A tissue experiments were assayed for IL-1Ra, AAT, IL-10, or albumin via in-house and commercial ELISA kits (R&D Systems, abcam), contracted services for multiplex ELISAs (Myriad), or chemiluminescent multiplex assay kits (Aushon).

When monolayer C3A cells were co-incubated with both IL-1β and IL-6, a synergistic response was observed towards increased IL-1Ra secretion, which was further increased at 54 h (FIG. 9). Albumin levels were decreased in response to IL-1 β and IL-6 in these same monolayer samples at 54 h (FIG. 11). However, when monolayer C3A cells were incubated with IL-1 β or IL-6 only, secretion of IL-1Ra did not increase above the level of the untreated controls.

AAT, in general, was secreted at concentrations nearly 1,000-fold higher than IL-1Ra in monolayer C3A cells. However, there was no apparent effect on AAT secretion when exposed to either IL-6, IL-1β, or their combination (FIG. 10). There was a time-dependent increase in AAT concentrations under all treatment conditions.

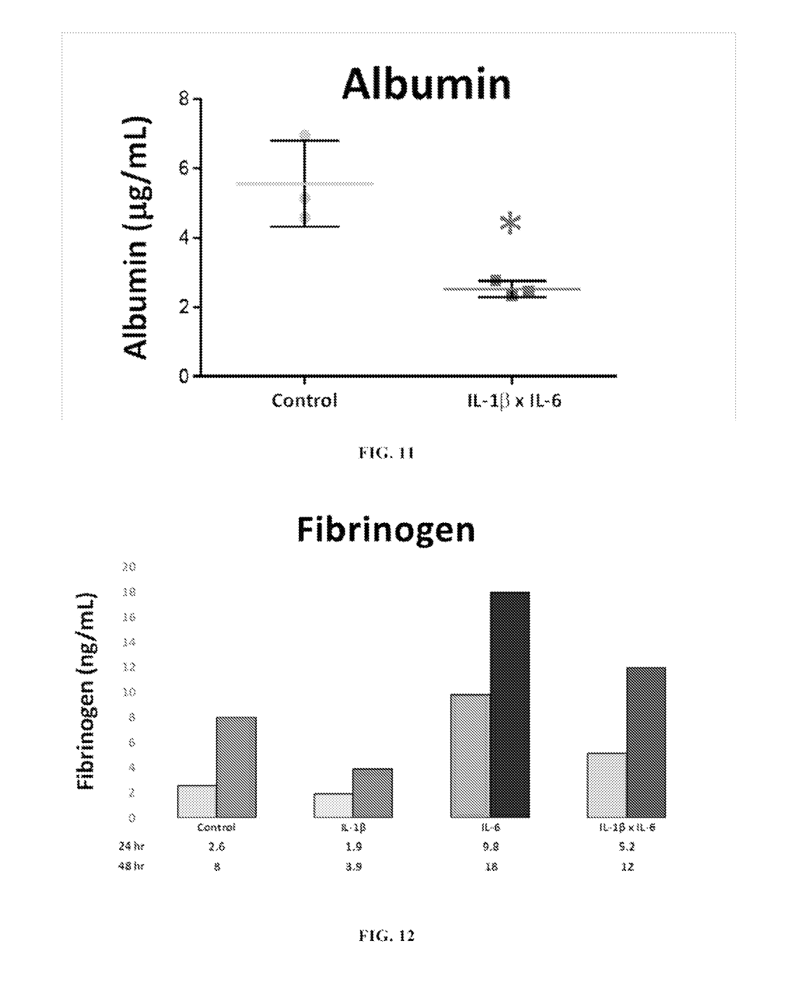

Fibrinogen was observed to increase predominantly in IL-6-treated monolayer C3A cultures. Expression was abrogated by addition of IL-1β (FIG. 12). α-2 macroglobulin also increased in response to IL-6 but not in response to IL-1β (FIG. 13).

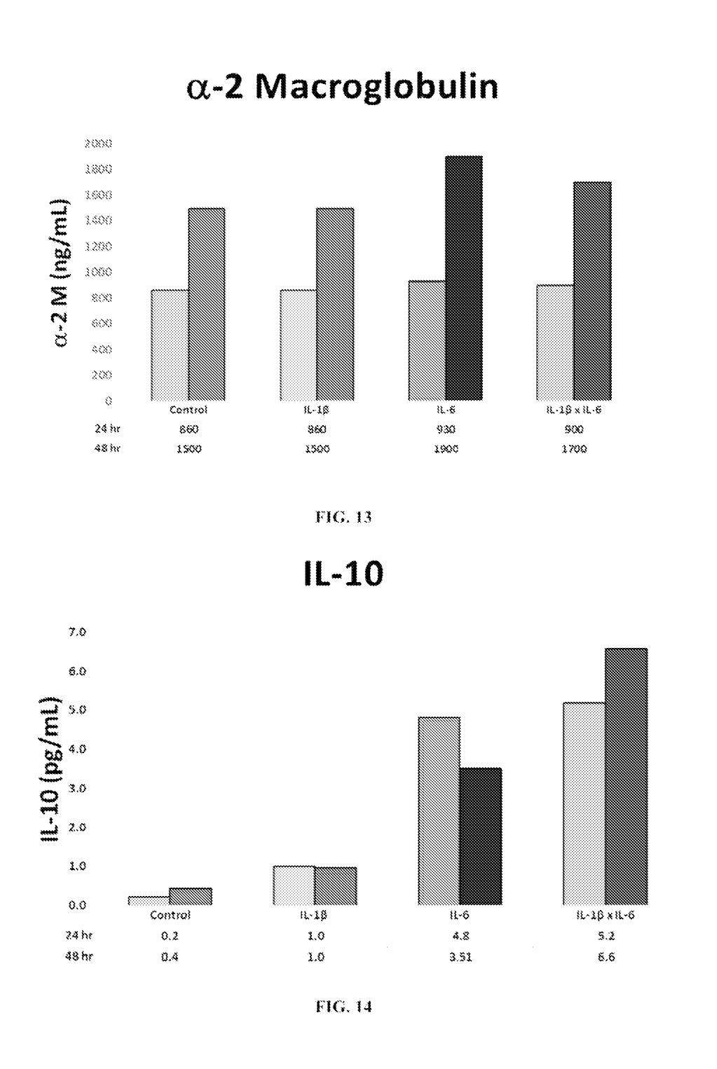

When monolayer C3A cells were incubated with 10 ng/mL IL-1β only, 10 ng/mL IL-6 only, or in combination, the secretion of IL-10 increased above the controls, and further increased at 48 hr. The dosing of IL-6 had a greater effect on IL-10 secretion (FIG. 14).

An increase in IL-1Ra (FIG. 15), but not in AAT (FIG. 8), was also observed when C3A tissue was incubated with IL-1β and IL-6 (10 ng/mL each) for 24 h. Sample sizes were increased to 6 replicates to help control for variability due to an inability to normalize results to cell counts after dosing.

TNF-α secretion by monolayer C3A cells increased in response to IL-1β and IL-6 in combination (FIG. 17). A similar increase was seen in C3A tissue (data not shown).

TNF-α (1, 10, or 100 ng/mL) alone did not increase secretion of IL-1Ra by monolayer C3A cells, although there was an observed decrease in secretion of IL-1Ra at the highest dose (100 ng/mL) (FIG. 18). IL-1β (10 ng/mL) alone and combination with and TNF-α (10 ng/mL each), increased secretion of IL-1Ra (FIG. 18). Once again, there was no effect on AAT secretion in any dose group (data not shown).

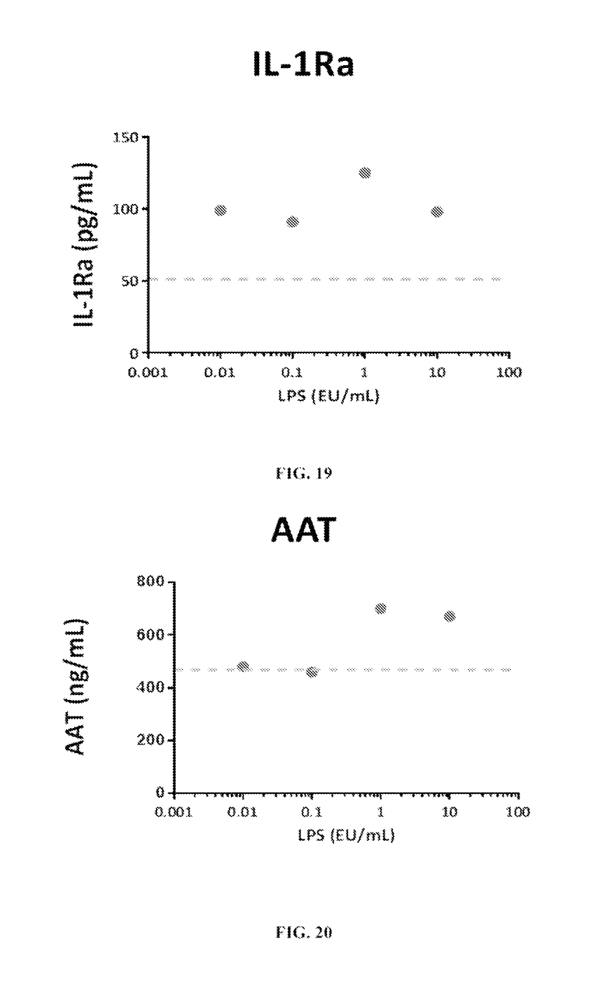

Direct exposure of monolayer C3A cells to LPS increased IL-1Ra secretion approximately 2-fold at all concentrations evaluated (0.01, 0.1, 1, and 10 EU/mL) (FIG. 19). AAT levels were increased only at higher concentrations (1 and 10 EU/mL) (FIG. 20).

C-reactive protein was below the lower level of quantitation of the assay (0.012 ng/mL). Haptoglobin levels did not change significantly with any treatment (data not shown).

Other factors observed to be predominantly increased in IL-6 treated C3A cells in monolayer include the following: Fibrinogen (˜6-fold vs. control (24, 48 hr)), IL-10 (˜10-fold vs. control (24 hr)), IL-18 (˜9-fold vs. control (24, 48 hr)), Monocyte chemotactic protein 1 (MCP-1) (˜15-fold vs. control (24, 48 hr), ˜20-fold with IL-1β vs. control (24, 48 hr), Tumor necrosis factor-beta (TNF-α) (˜12-fold vs. control (24, 48 hr)), Granulocyte-colony stimulating factor (G-CSF) (˜50- to 75-fold vs. control (24 hr, 48 hr), ˜300- and 500-fold with IL-6 vs. control (24 hr, 48 hr)), Stem cell factor (SCF) (˜100-fold vs. control (24 hr, 48 hr)), IL-7 (˜10- and 14-fold vs. control (24 hr, 48 hr)), IL-8 (˜2,000-fold vs. control (24 hr, 48 hr) and TNFα (˜25-fold vs. control (24 hr, 48 hr))

Other factors observed to be predominantly increased in IL-1β and IL-6 treated C3A cells in monolayer include Intracellular adhesion molecule 1 (ICAM-1) (˜9-fold vs. control (24 hr)) and Transthyretin (˜3-fold decrease vs. control (48 hr)).

Figure Legends

FIG. 9: Monolayer C3A cell (1.3×10⁵ cells/cm) secretion of IL-1Ra is upregulated in the combined presence of IL-6 and IL-1β and increases with exposure time. Results are mean±SD, n=3 biological replicates (left-24 h, right-54 h for each pair of bars).

FIG. 10: Monolayer C3A cell (1.3×10⁵ cells/cm) secretion of AAT increases with time, but is not affected by exposure to IL-6 or IL-1β individually or in combination. Results are mean±SD, n=3 biological replicates (left-24 h, right-54 h for each pair of bars).

FIG. 11: Monolayer C3A cell (1.3×10⁵ cells/cm) secretion of albumin is downregulated at 54 h in the combined presence of IL-6 (10 ng/mL) and IL-1β (10 ng/mL) (*p=0.047), as expected for this APP. Results are mean±SD, n=3 replicates.

FIG. 12: Monolayer C3A cell (1.3×10⁵ cells/cm²) secretion of fibrinogen was increased by IL-6 and abrogated by IL-1β. Results are single replicates of pooled triplicate wells.

FIG. 13: Monolayer C3A cell (1.3×10⁵ cells/cm²) secretion of α-2 Macroglobulin (α-2M) appeared modestly upreguated by IL-6 alone. Results are single replicates of pooled triplicate wells.

FIG. 14: Monolayer C3A cell (1.3×10⁵ cells/cm²) secretion of IL-10 is upregulated in the presence of IL-1β or IL-6, and is more driven by IL-6. Results are single replicates of pooled triplicate wells (left-24 h, right-48 h for each pair of bars).

FIG. 15: C3A tissue (not normalized to cell number) secretion of IL-1Ra is also upregulated at 24 h in the combined presence of IL-6 (10 ng/mL) and IL-1β (10 ng/mL) (*p=0.007). Results are mean±SD, n=6 biological replicates.

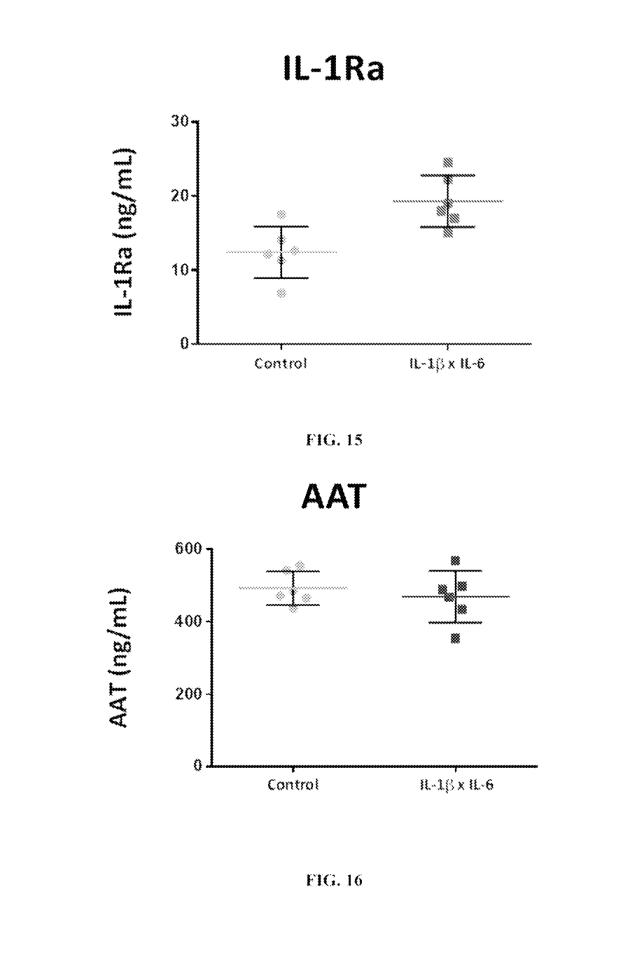

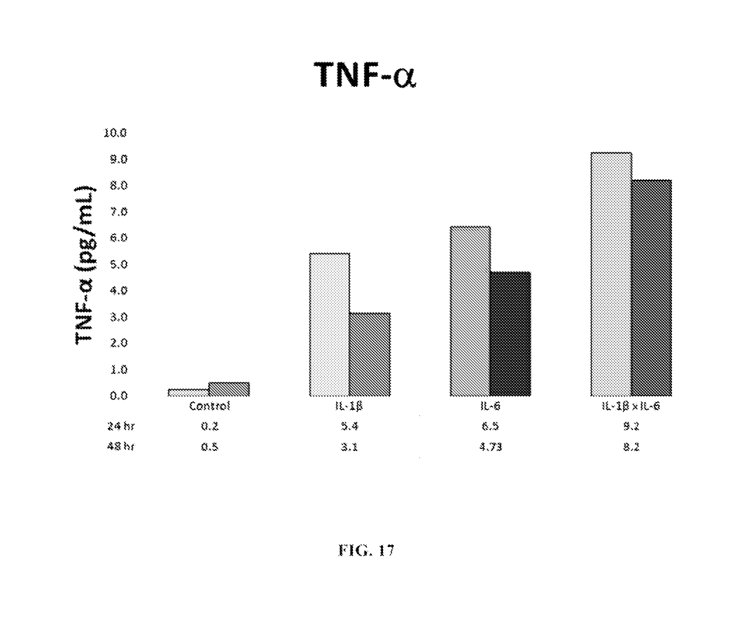

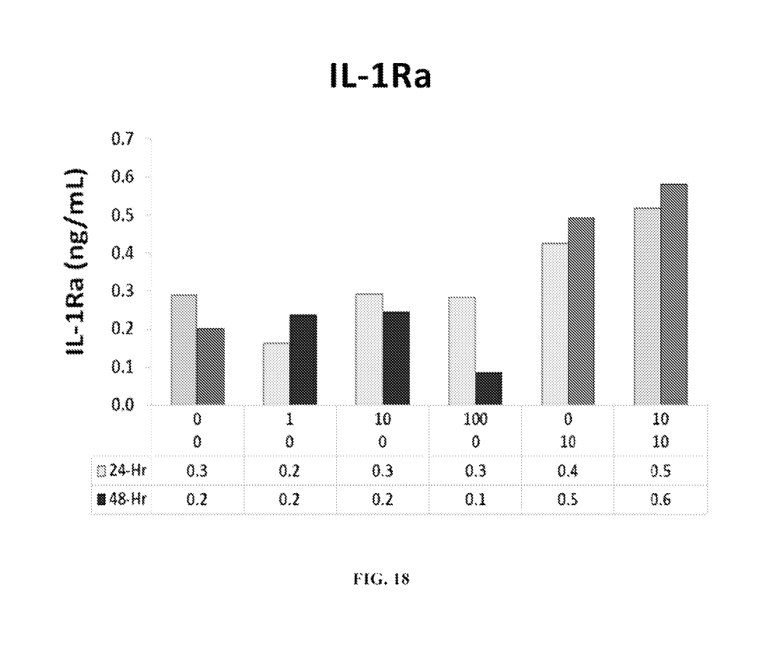

FIG. 16: C3A tissue (not normalized to cell number) secretion of AAT is not upregulated at 24 h in the combined presence of IL-6 (10 ng/mL) and IL-1β (10 ng/mL) (p=0.51). Results are mean±SD, n=6 biological replicates.

FIG. 17: Monolayer C3A cell (2.6×10⁵ cells/cm²) secretion of TNFα is upregulated in the presence of IL-1β or IL-6. Results are single replicates of pooled triplicate wells (left-24 h, right-48 h for each pair of bars).

FIG. 18: Monolayer C3A cell (2.6×10⁵ cells/cm²) secretion of IL-1Ra is upregulated in the presence of IL-1β (10 ng/mL) and combination of IL-1β and TNF-α (10 ng/mL each), but not TNF-α alone. Results are single replicates of pooled triplicate wells.

FIG. 19: Monolayer C3A cell (1.3×10⁵ cells/cm²) secretion of IL-1Ra is upregulated at 24 h in the presence of LPS. Results are single replicates of pooled triplicate wells (green line indicates untreated control response).

FIG. 20: Monolayer C3A cell (1.3×10⁵ cells/cm²) secretion of AAT is upregulated at 24 h in the presence of higher concentrations of LPS. Results are single replicates of pooled triplicate wells (green line indicates untreated control response).

Discussion

In these studies, the ability of C3A cells in both monolayer and C3A tissue to respond to pro-inflammatory cytokines and key mediators of the acute phase response, IL-1β, IL-6 and TNF-α, and to LPS was demonstrated.

In both monolayer and C3A tissue, the C3A cells respond to these inflammatory mediators, found elevated in AILD patients, by upregulated and/or constitutive expression of anti-inflammatory APPs.

As is characteristic of an acute phase response, the effects of IL-6 and IL-1β vary dependent upon the resulting factor, and can be inhibitory (e.g. fibrinogen) or enhancing (e.g. IL-1Ra) of each other. Reduced albumin production, in parallel with increased IL-1Ra secretion, is also consistent with an acute phase response.

Exogenous AAT and IL-1Ra have been shown to suppress pro-inflammatory cytokine synthesis by interference with TNF-α and IL-1β pathways and enhancement of IL-10 production, the latter of which has broad anti-inflammatory properties. It is not clear from these studies whether increased IL-10 production by C3A cells results directly from IL-6 exposure or autocrine effects of IL-1Ra.

C3A cells produced low, yet elevated, levels of TNF-α in response to IL-6, IL-1β and the combination. However TNF-α did not significantly impact APP expression except when dosed 10,000-fold higher than measured in culture.

Reduction of pro-inflammatory cytokines and increases in anti-inflammatory APPs in response to elevated cytokines and LPS in AILD patients may contribute to resolution of inflammation by the therapeutic system.

Conclusions

C3A cells secrete anti-inflammatory factors both constitutively and in response to co-incubation with IL-1β and IL-6. Their response is dynamic, exhibiting temporal and dose-dependent secretion of anti-inflammatory mediators, IL-1Ra and AAT. Additionally, C3A cells upregulate IL-1Ra and AAT in response to LPS.

An inflammation resolution response may represent one of the multiple mechanisms for the therapeutic benefit resolution in AILD patients treated with the therapeutic system.

### Example 4

### Ex Vivo and In Vitro Models Support that C3A Cell-Secreted Factors Help Reduce Inflammation and Neutrophil/Macrophage Dysfunction Associated with Alcoholic Liver Disease to Restore Immune Homeostasis

Alcoholic hepatitis (AH) patients have high levels of pro-inflammatory cytokines and compromised immune cell function, in part due to increased gut permeability from chronic alcohol consumption and translocation of bacteria/endotoxin to the circulation. Although the mechanisms for progression to systemic inflammatory response syndrome (SIRS) and multi-organ failure are not fully understood, the associated increase in mortality is well documented.

The purpose of these studies was to evaluate the potential for the treatment system disclosed herein having C3A cells of the invention to restore immune homeostasis using ex vivo and in vitro models of innate immune cell function.

Plasma samples collected from 7 system-treated (for treatment of Acute Chronic Liver Failure) and 2 disease severity-matched control subjects were measured for cytokines prior to and 24 h after start of treatment. Oxidative burst and phagocytic capacity were measured in healthy control neutrophils treated with subject plasma collected before, during, and after treatment.

THP-1 monocytic cells were induced to adhere, polarized to a pro-inflammatory (M i) macrophage phenotype, and measured for cytokine production and phagocytic capacity in the presence/absence of C3A cell conditioned media (CM).

IL-1β, IL-6 and TNFα levels in subject plasma all trended downward 24 h after treatment, suggesting a shift from pro-inflammatory TH1-like profile to anti-inflammatory TH2 profile. Oxidative burst was significantly higher than control plasma for neutrophils treated with pre-system-treatment subject plasma and trended downward while on ELAD treatment and thereafter. Phagocytosis of FITC-labeled E. coli was lowest in neutrophils treated with pre-ELAD-treatment plasma and after 24 h of ELAD-treatment and increased in neutrophils treated with plasma from ELAD-treated subjects at the end of treatment and at 30-d follow-up.

Treating pro-inflammatory (MI) THP-1 cells with CM reduced inflammatory cytokine secretion (IL-1β, IL-6, and TNFα), phagocytosis was not restored within 48 h.

It was previously reported that C3A cells produce anti-inflammatory protein IL-1Ra in response to exposure to pro-inflammatory cytokines IL-1β and IL-6, and that IL-1Ra concentrations increased in plasma of system-treated subjects during treatment in a case study (n=3). These current studies demonstrate that C3A cells restore immune homeostasis in ex vivo and in vitro models of innate immune cell function and suggest a potential mechanism by which treatment may provide benefit.

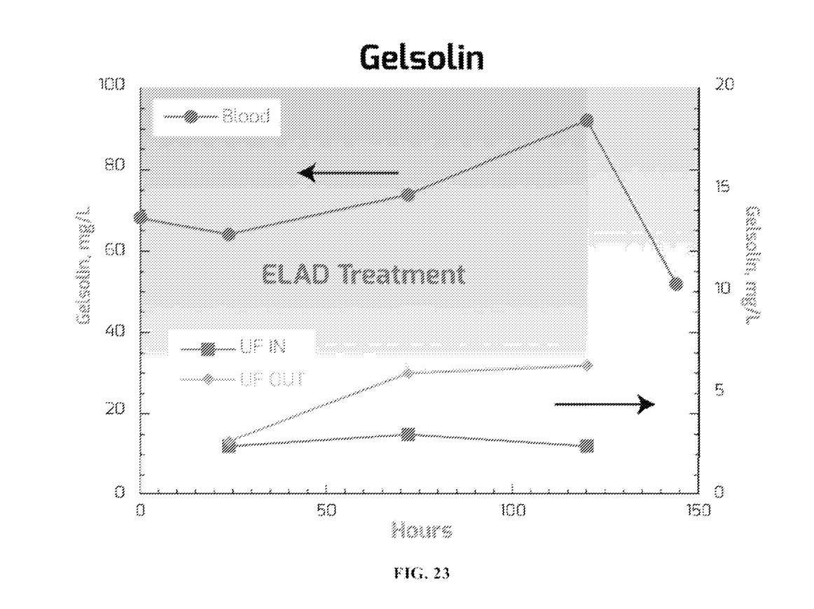

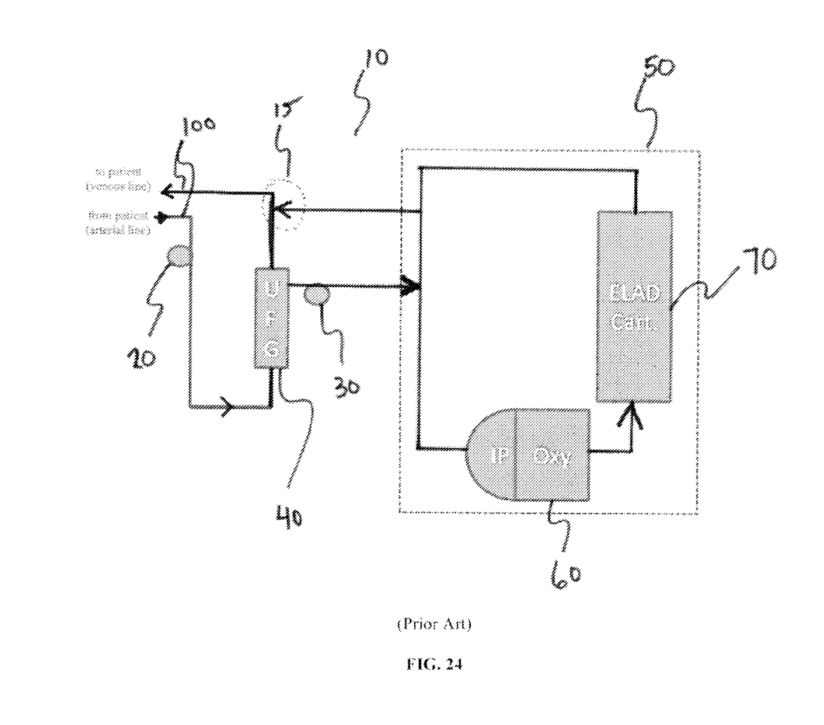

Gelsolin levels in subject plasma trended significantly upward 24 h after treatment as shown in FIG. 23.

Figure Legends

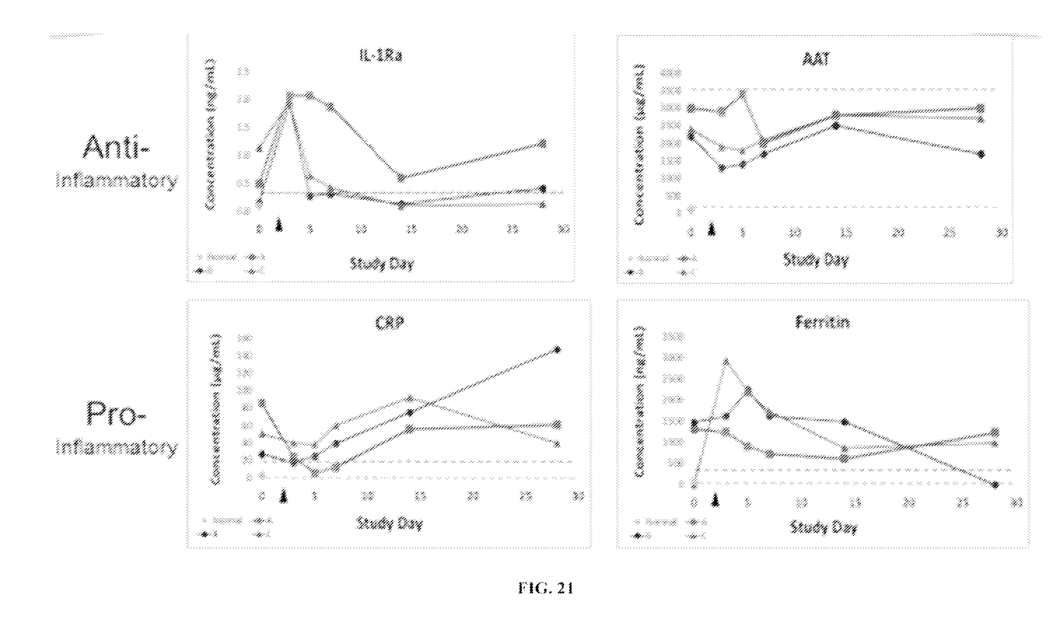

FIG. 21: Selected protein levels in subject plasma during treatment with the treatment system.

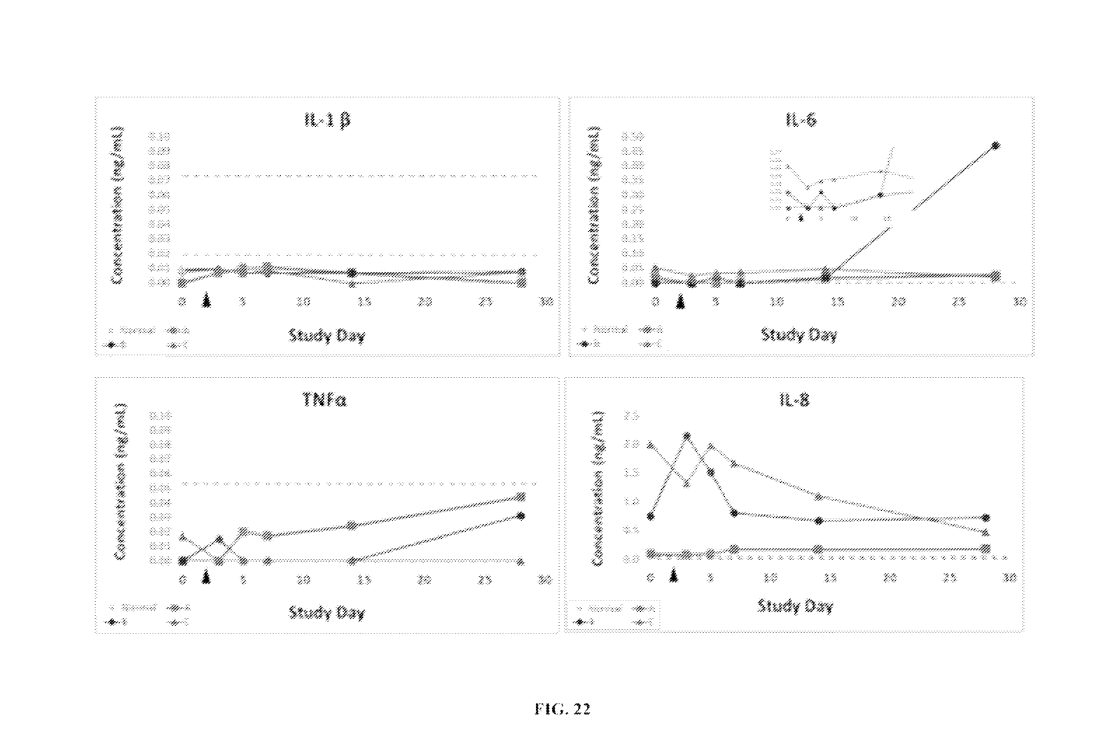

FIG. 22: Selected protein levels in subject plasma during treatment with the treatment system.

FIG. 23: Levels of gelsolin in subject plasma during treatment with the treatment system.

The present invention has been illustrated and described in detail with reference to particular embodiments by way of example only, and not by way of limitation. Those of skill in the art will appreciate that various modifications to the described exemplary embodiments are within the scope and contemplation of the present disclosure. Therefore, it is intended that the invention be considered as limited only by the scope of the appended claims.

1. A composition for inducing an anti-inflammatory response in a cell, the composition comprising one or more pro-inflammatory molecules, wherein the anti-inflammatory response comprises increased expression of anti-inflammatory factors.

2. The composition of claim 1, wherein the cell is a eukaryotic cell.

3. The composition of claim 1, wherein the cell is a mammalian cell.

4. The composition of claim 3, wherein the cell is a human cell.

5. The composition of claim 1, wherein the cell is a hepatocyte.

6. The composition of claim 1, wherein the cell is a recombinantly engineered cell.

7. The composition of claim 1, wherein the cell is a hepatoblastoma-derived cell.

8. The composition of claim 7, wherein the cell is a HepG2 cell or a C3A cell.

9. The composition of claim 8, wherein the cell is a clonal derivative from a parental C3A cell line.

10. The composition of claim 1, wherein the pro-inflammatory molecules comprise one or more cytokines, damage-associated molecular pattern molecules (DAMPs), or pathogen-associated molecular pattern molecules (PAMPs)

11. The composition of claim 1, wherein the pro-inflammatory molecules comprise one or more of Tumor necrosis factor alpha (TNF-α), Interleukin-1 (IL-1), Interleukin-5 (IL-5), Interleukin-6 (IL-6), Interleukin-8 (IL-8), Interleukin-11 (IL-11), Interleukin-12 (IL-12), Interleukin-17 (IL-17), Interleukin-18 (IL-18), Interleukin-1 beta (IL-1β), Monocyte chemotactic protein-1 (MCP-1), Macrophage inflammatory protein 1-alpha (MIP-1α), Macrophage inflammatory protein 1-beta (MIP-1β), Interferon gamma (IFN-γ), Granulocyte-macrophage colony-stimulating factor (GM-CSF), lymphotactin, fractalkine, or any combination thereof.

12. The composition of claim 1, wherein the composition is derived from blood of a subject having a disease.

13. The composition of claim 12, wherein the disease is an inflammatory disease.

14. The composition of claim 13, wherein the inflammatory disease is a liver disease selected from the group consisting of cirrhosis, hepatitis and fatty liver disease.

15. The composition of claim 13, wherein the disease is an autoimmune disease or autoinflammatory disease.

16. The composition of claim 1, wherein the anti-inflammatory factors comprise one or more of Alpha-1-Antitrypsin (AAT), Interleukin-1 receptor antagonist (IL-1Ra), Interleukin-4 (IL-4), Interleukin-10 (IL-10), Interleukin-13 (IL-1β), Interferon alpha (IFN-α), Gelsolin, Transforming Growth Factor beta (TGF-β), or any combination thereof.

17. The composition of claim 16, wherein the expression of the anti-inflammatory factors are increased by a factor of at least 2.0, 5.0, 10, 25, 50, 100, 250, 500, 1000 or greater.

18. The composition of claim 1, wherein the cell is contacted in-vitro.

19. The composition of claim 18, wherein the cell is adhered to a solid substrate.

20. The composition of claim 18, wherein the cell is embedded in a semi-solid substrate.

21. The composition of claim 1, wherein the cell is contacted in-vivo.

22. The composition of claim 1, wherein the composition further comprises a eukaryotic cell.

23. The composition of claim 1, wherein the eukaryotic cell is a mammalian cell.

24. The composition of claim 3, wherein the eukaryotic cell is a human cell.

25. The composition of claim 1, wherein the eukaryotic cell is a hepatocyte.

26. The composition of claim 1, wherein the eukaryotic cell is a recombinantly engineered cell.

27. The composition of claim 1, wherein the eukaryotic cell is a hepatoblastoma-derived cell.

28. The composition of claim 7, wherein the eukaryotic cell is a HepG2 cell or a C3A cell.

29. The composition of claim 8, wherein the eukaryotic cell is a clonal derivative from a parental C3A cell line.

30. A method of inducing an anti-inflammatory response, or inhibiting an inflammatory response, in a cell comprising contacting the cell with the pro-inflammatory composition according to claim 1, wherein the anti-inflammatory response comprises increased expression of anti-inflammatory factors.

31-52. (canceled)

53. A method of treating a disease or disorder in a subject comprising administering a composition comprising anti-inflammatory factors to the subject, thereby treating the disease or disorder.

54. The method of claim 53, wherein the disease or disorder is an inflammatory disease.

55. The method of claim 54, wherein the disease is an autoimmune disease or autoinflammatory disease.

56. The method of claim 53, wherein the disease is a liver disease selected from the group consisting of cirrhosis, hepatitis and fatty liver disease.

57-64. (canceled)

65. A qualified C3A cell line derived from a parental C3A cell line, wherein cells of the cell line exhibit increased expression of anti-inflammatory mediator proteins α-1-antitrypsin (AAT) and Interleukin-1 receptor antagonist (IL-1Ra) in response to lipopolysaccharide (LPS) or pro-inflammatory cytokines Interleukin-6 (IL-6) and Interleukin-1 beta (IL-1β).

66. The cell line of claim 65, wherein the increase of expression of AAT or IL-1Ra is by a factor of at least 2.0, 5.0, 10, 25, 50, 100, 250, 500, 1000 or greater as compared to a cell not contacted with LPS or IL-6 and IL-1β.

67. The cell line of claim 65, wherein the cells further exhibit increased expression of gelsolin.

68. The cell line of claim 67, wherein the expression of gelsolin is by a factor of at least 2.0, 5.0, 10, 25, 50, 100, 250, 500, 1000 or greater as compared to a cell not contacted with LPS or IL-6 and IL-1β.
